(19) United States
(12) Patent Application Publication
(10) Pub. No.: US 2018/0291114 A1
(43) Pub. Date: Oct. 11, 2018

(54) RECOMBINANT BI-SPECIFIC POLYPEPTIDE FOR COORDINATELY ACTIVATING TUMOR-REACTIVE T-CELLS AND NEUTRALIZING IMMUNE SUPPRESSION

(71) Applicant: UNIVERSITY OF MARYLAND, BALTIMORE COUNTY, Baltimore, MD (US)

(72) Inventors: Suzanne Ostrand-Rosenberg, Columbia, MD (US); Darryl L. Carter, White Marsh, MD (US)

(21) Appl. No.: 16/007,035

(22) Filed: Jun. 13, 2018

Related U.S. Application Data
(63) Continuation-in-part of application No. PCT/US2016/066842, filed on Dec. 15, 2016.
(60) Provisional application No. 62/268,682, filed on Dec. 17, 2015.
(60) Provisional application No. 62/355,422, filed on Jun. 28, 2016.
(60) Provisional application No. 62/538,053, filed on Jul. 28, 2017.

(51) Int. Cl. C07K 16/46; C07K 16/30; C07K 16/28; A61K 39/395

(52) U.S. Cl. CPC C07K 16/468; C07K 16/3053; A61P 35/00; A61K 39/39558; A61K 39/3955; C07K 16/2809

(57) ABSTRACT

The present invention relates generally to the field of generating fusion proteins to be used in cancer therapy, and more specifically, a bispecific T-Cell engager recombinant polypeptide comprising an antibody, fragment thereof, or single chain variable fragment that binds to CD3 of a T-cell antigen receptor and an antibody, fragment thereof, or single chain variable fragment that binds to Programmed Death Ligand 1 (PDL1) on a cancerous tumor cell to counteract the immune tolerance of cancer cells.

### CROSS-REFERENCE TO RELATED APPLICATIONS

This application is a Continuation-in-Part and claims priority to PCT International Application No. PCT/US2016/066842 filed on Dec. 15, 2016 which in turn claims priority to U.S. Provisional Patent Application No. 62/268,682, filed on Dec. 17, 2015, U.S. Provisional Patent Application No. 62/355,422, filed Jun. 28, 2016; and also claims priority to U.S. Provisional Patent Application No. 62/538,053, filed Jul. 28, 2017, the contents of which are hereby incorporated by reference herein for all purposes.

### BACKGROUND OF THE INVENTION

### Technical Field

The present invention relates generally to the field of fusion proteins to be used to stimulate an immune response, and more specifically, a bispecific T-Cell engager recombinant polypeptide comprising a moiety that binds to CD3 on a T-cell and a moiety that binds to Programmed Death Ligand 1 (PDL1) on a tumor cell to counteract the immune tolerance of cancer cells.

### Related Art

The immune system provides the human body with a means to recognize and defend itself against microorganisms and substances recognized as foreign or potentially harmful. While passive immunotherapy of cancer with monoclonal antibodies and passive transfer of T-cells to attack tumor cells have demonstrated clinical efficacy, the goal of active therapeutic vaccination to induce these immune effectors and establish immunological memory against tumor cells has remained challenging. Several tumor-specific and tumor-associated antigens have been identified, yet these antigens are generally weakly immunogenic and tumors employ diverse mechanisms to create a tolerogenic environment that allows them to evade immunologic attack.

Strategies to overcome such immune tolerance and activating robust levels of antibody and/or T-cell responses hold the key to effective cancer immunotherapy.

While existing bi-specific T-cell engagers have clinical efficacy, they do not reduce immune suppression and therefore are not therapeutic in all cancer patients. Thus, there is a need to develop a bi-specific T-cell engager that activates T-cells, induces cytotoxicity and kills tumor cells despite the presence of immune suppression mediated by PD1.

### SUMMARY OF THE INVENTION

The present invention provides for a bispecific T-Cell engager (BiTE) recombinant protein useful in a variety of therapeutic methods for the treatment of cancer. Bi-specific T-cell engagers (BiTE) are genetically engineered recombinant proteins that simultaneously activate T-cells and induce the T cells to become cytotoxic through the T-cell receptor/CD3 complex and target the activated T-cells to tumor cells. The present invention provides methods of reducing growth and/or killing of cancer cells by counteracting immune tolerance of cancer cells, wherein T-cells remain active and inhibit the recruitment of regulatory cells that are known to suppress the immune system's response to the tumor. The present invention is also designed to concurrently activate naïve T-cells through the binding of anti-CD3, while binding to target tumor cells or other cells expressing the inhibitory ligand PDL1.

In one aspect, the present invention provides for an anti-CD3/anti PDL1 bispecific fusion protein comprising an anti-CD3 antibody, fragments thereof, or single chain variable fragments that binds with CD3 on a T-cell and an anti-PDL1 antibody, fragments thereof, or single chain variable fragments that binds to Programmed Death Ligand 1 (PDL1) on a tumor cell to counteract the immune tolerance of cancer cells, wherein the anti-CD3 antibody , fragment thereof, or single chain variable fragment and the anti-PDL1 antibody, fragment thereof, or single chain variable fragment are linked by an amino acid spacer of sufficient length of amino acid residues so that both moieties can successfully bind to their individual target.

In the alternative, the anti-CD3 moiety and the anti-PDL1 moiety that counteract immune tolerance of cancer cell may be bound directly to each other without a linker. The anti-CD3/anti-PDL1 bispecific fusion protein of the present invention is useful for binding to a cancer cell receptor (PDL1) and reducing the ability of cancer cells to avoid an immune response, while concurrently activating and directing T-cells (CD3) to the targeted tumor cell and inducing death of such cancer cell.

The present invention is based on preparing bispecific proteins by expression of polynucleotides encoding the bispecific proteins that counteract or reverse immune tolerance of cancer cells and simultaneously activate T-cells. Cancer cells are able to escape elimination by chemotherapeutic agents or tumor-targeted antibodies via specific immunosuppressive mechanisms in the tumor microenvironment and such ability of cancer cells is recognized as immune suppression. By counteracting tumor-induced immune suppression, the present invention provides effective compositions and methods for cancer treatment, optionally in combination with another existing cancer treatment. The present invention provides strategies to counteract tumor-induced immune suppression by activating and leveraging T-cell-mediated adaptive antitumor immunity against resistant cancer cells.

In another aspect, the present invention provides for a method of treating cancer and/or killing cancerous tumor cells, the method comprising:
administering to a subject an anti-CD3/anti-PDL1 bispecific fusion protein comprising an anti-CD3 antibody ,fragments thereof, or single chain variable fragments that binds to a CD3 receptor on a T-cell and an anti PDL1 antibody, fragments thereof, or single chain variable fragments that binds to PDL1 on a tumor cell, wherein the binding to CD3 on CD4⁺ or CD8⁺ T-cell activates the T-cells that are cytotoxic for PDL1⁺ tumor cells, thereby inducing death of the PDL1⁺ tumor cells.

Notably the activated T-cells cells have been found to be cytotoxic for PDL1⁺ human melanoma, chronic myelogenous leukemia (CML), breast cancer, and non-small cell lung cancer cells (NSCLC).

The amino acid residues for the variable regions of the light and heavy chains of the anti-PDL1 antibody are SEQ ID NOs. 5 and 6, respectively and the variable region of the light and heavy chains of the anti-CD3 antibody are SEQ ID NOs. 7 and 8, respectively. The linker may include at least one of the amino acid residues SEQ ID NOs. 12, 14 or 16. The variable region of the heavy or light chain of the anti-PDL1 antibody may be linked to the variable region of the light or heavy chain of an anti-CD3 antibody. Preferably, the variable region of the heavy chain of the anti-PDL1 antibody is linked to variable region of the heavy chain of an anti-CD3 antibody.

In one aspect, the present invention provides for genes encoding for a bispecific comprising an anti-CD3 antibody or fragments thereof and an anti-PDL1 antibody or fragments thereof for treating cancer in a human subject for expression in a human subject. Preferably the genes comprise sequences selected from SEQ ID NOs: 1 to 4, that being, the variable regions of the heavy (VH) and light (VL) chains of antibodies that bind to the CD3 component of the T-cell antigen receptor and to PDL 1.

In yet another aspect, the present invention provides for a method of treating cancer with an anti-CD3/anti-PDL1 bispecific fusion protein in a subject, the method comprising:
providing at least one recombinant vector comprising nucleotide sequences that encode for an anti-CD3 antibody or fragments thereof and an anti-PDL1 antibody or fragments thereof wherein the nucleotide sequences comprise SEQ ID NOs: 1 to 4; and administering the recombinant vector to the subject under conditions such that said nucleotide sequences are expressed at a level which produces a therapeutically effective amount of the encoded bispecific fusion protein in the subject, wherein the expressed amino acid residues for the bispecific fusion protein comprises SEQ ID NOs. 7 and 8 for the light and heavy chains of the anti-CD3 antibody respectively and SEQ ID NOs. 5 and 6 for the light and heavy chains of the anti-PDL1 antibody respectively.

In yet another aspect, the present invention provides a recombinant host cell transfected with a polynucleotide that encodes an anti-CD3/anti-PDL1 bispecific fusion protein of the present invention.

In a still further aspect, the present invention contemplates a process of preparing a bispecific fusion protein of the present invention comprising:
transfecting a host cell with polynucleotide sequences that encode a bispecific fusion protein to produce a transformed host cell, wherein the polynucleotide sequences encode an anti-CD3 antibody and an anti-PDL1 antibody, wherein the nucleotide sequences comprise SEQ ID NOs: 1 to 4; and
maintaining the transformed host cell under biological conditions sufficient for expression of the bispecific fusion protein.

In yet a further aspect, the present invention provides a method of preventing or treating a neoplastic disease. The method includes administration to a subject in need thereof a bispecific fusion protein of the invention in combination with another anticancer therapy, in one aspect, the anticancer therapy includes a chemotherapeutic molecule, antibody, small molecule kinase inhibitor, such as tyrosine kinase inhibitors (TKIs), hormonal agent or cytotoxic agent. The anticancer therapy may also include ionizing radiation, ultraviolet radiation, cryoablation, thermal ablation, or radiofrequency ablation.

In another aspect, the present invention provides for a method of preparing a therapeutically active anti-CD3/anti-PDL1 bispecific fusion protein comprising an anti-CD3 moiety and an anti-PDL1 moiety, the method comprising;
preparing a nucleotide sequence of the anti-CD3/anti-PDL1 bispecific fusion protein; cloning the nucleotide sequence of the anti-CD3/anti-PDL1 bispecific fusion protein in a host cell capable of transient or continued expression;
growing the host cell in a media under suitable conditions for growing and allowing the host cell to express the cloned anti-CD3/anti-PDL1 bispecific fusion protein; and subjecting the expressed bispecific fusion protein to purification and optionally checking the bi-specific binding capabilities of the anti-CD3/anti-PDL1 bispecific fusion protein to its targets.

Such tests may include in-vitro test such as ELISA or NK/T-cell binding assays to validate bi-functional target binding or immune cell stimulation.

Notably once the anti-CD3/antiPDL1 bispecific fusion protein demonstrates the desired bi-specificity, such anti-CD3/antiPDL1 bispecific fusion protein is selected for sub-cloning into a stable cell line for larger scale expression and purification. Such stable cell lines are previously disclosed, such as a mammalian cell line, including but not limited to HEK293, CHO or NSO or a bacterial cell line, including but not limited to E. coli, or in yeast, including but not limited to saccharomyces cerevisiae.

In a still further aspect, the present invention provides for use of an anti-CD3/anti-PDL1 bispecific fusion protein comprising an anti-CD3 moiety that binds to CD3 on a T-cell and an anti-PDL1 moiety that binds to Programmed Death Ligand 1 (PDL1) on a tumor cell in a medicament for the treatment of cancer.

In another aspect, the present invention provides for a method of killing cancerous tumor cells, the method comprising:
contacting a cancerous tumor cell with an anti-CD3/antiPDL1 bispecific fusion protein comprising an anti-CD3 antibody or fragments thereof that binds to a CD3 receptor on a T-cell and an anti-PDL1 antibody or fragments thereof that binds to Programmed Death Ligand 1 (PDL1⁺) on a tumor cell, wherein the binding to the CD3 receptor on the T-cell activates the T-cells that are cytotoxic for a PDL1⁺ tumor cell, such as activating CD4⁺ and CD8⁺ T-cells and thereby inducing increased death of the PDL1⁺ tumor cell.

In yet another aspect, the present invention provides a method of preparing an anti-CD3/anti-PDL1 bispecific fusion protein, the method comprising:
transfecting a host cell with polynucleotide sequences that encode the anti-CD3/anti-PDL1 bispecific fusion protein to produce a transformed host cell, wherein the polynucleotide sequences encode an anti-CD3 antibody, fragment thereof, or single chain variable fragment and an anti-PDL1 antibody, fragment thereof, or single chain variable fragment and having a nucleotide sequence of SEQ ID NO: 9; and
maintaining the transformed host cell under biological conditions sufficient for expression of the bispecific fusion protein have an amino acid sequence of SEQ ID NO: 10.

Other features and advantages of the invention will be apparent from the following detailed description, drawings and claims.

### BRIEF DESCRIPTION OF THE FIGURES

FIG. 1 (A, B and C) showa CHO cells transfected with the anti-CD3/anti-PDL1 fusion protein produce ˜1.2 μg protein/10⁷ cells/48 hrs. FIG. 1A shows that the anti-CD3/anti-PDL1 fusion protein (referred to as BiTE in all figures) of the present invention is a ˜55 kD secreted protein. CHO cells were transfected with nucleotide sequences encoding the anti-CD3/anti-PDL1 fusion protein and were incubated at 37° C. 5% CO₂ in serum-free HL1 or serum-containing IMDM medium. Supernatants were collected, concentrated using 10 kD spin columns, electrophoresced by SDS-PAGE, and western blotted with an anti-His antibody. FIG. 1B shows the construct used to transfect CHO cells. FIG. 1C shows 1×10⁷ transfectants were incubated in 100 ml of serum-free HL-1 medium for 48 hrs. Supernatants were collected and remaining cells were removed by centrifugation. Supernatants were concentrated to 1.5 ml using 10 kD spin columns. Concentrated supernatant was diluted 1:5 and 1:10 with PBS and slot blotted along with dilutions of a control his-tagged protein. Dried blots were probed with an anti-his antibody. The anti-CD3/anti-PDL1 fusion protein was quantified using Image J software.

FIG. 2 (A, B and C) shows that the anti-CD3/anti-PDL1 fusion protein (referred to as BiTE in all figures) of the present invention simultaneously and specifically binds to PDL1⁺ human tumor cells and CD3⁺ human T-cells. FIG. 2A shows the anti-CD3/anti-PDL1 fusion protein of the present invention binds specifically to PDL1⁺ human tumor cells. C8161 and MEL1011 melanoma cells were stained with antibodies to PDL1 (left panels). PDL1⁺ C8161 and PDL1⁻ MEL1011 cells were incubated with the anti-CD3/anti-PDL1 fusion protein of the present invention or an irrelevant control protein and stained with anti-His antibody to detect binding of the anti-CD3/anti-PDL1 fusion protein of the present invention (right panels). FIG. 2B shows that the anti-CD3/anti-PDL1 fusion protein of the present invention binds specifically to CD3⁺ human T-cells. PBMC from healthy human donors were stained with antibodies to CD4 and CD8 followed by addition of the anti-CD3/anti-PDL1 fusion protein or irrelevant protein, and then antibody to His to detect the binding of the anti-CD3/anti-PDL1 fusion protein. Some PBMC were first blocked with antibodies to CD3 to prevent binding of the anti-CD3/anti-PDL1 fusion protein. FIG. 2C shows that the anti-CD3/anti-PDL1 fusion protein simultaneously binds CD3 and PDL1. PBMC were stained for CD4 and CD8 as in panel B, and then stained with either the anti-CD3/anti-PDL1 fusion protein of the present invention or an irrelevant protein followed by soluble PDL1 (sPDL1). PDL1 binding was detected with antibody to PDL1. Data are representative of >3 independent experiments with multiple batches of the anti-CD3/anti-PDL1 fusion protein of the present invention.

FIG. 3 shows that the anti-CD3/anti-PDL1 fusion protein (BiTE) induces PBMC from healthy human donors to produce IFNγ in the presence of human melanoma (C8161) cells. Healthy donor PBMC (1.2×10⁶ cells) were incubated with the anti-CD3/anti-PDL1 fusion protein (6.5 ng/ml), C8161 human melanoma cells, and/or an irrelevant recombinant protein (Troy-Fc). Following a 48 hr incubation, supernatants were analyzed for IFNγ. PBMC to tumor cell ratio was 20:1. Data are representative of 1 of 2 independent experiments.

FIG. 4 (A and B) shows that the anti-CD3/anti-PDL1 fusion protein (BiTE) of the present invention activates human T-cells that are cytotoxic for PDL1⁺ human melanoma cells. PBMC from a healthy human donor were incubated at varying effector to target ratios with Cell Tracker violet-labeled PDL1⁺ C8161 human melanoma cells with or without (5.2 ng/ml). After a 48 hr incubation the cells were harvested. FIG. 4A shows flow profiles for CD25 and PD1 staining for representative samples; bar graph shows the average of three replicates per sample. One aliquot of cells was labeled with antibodies to CD8, CD25, and PD1. CD8⁺ T-cells were gated and analyzed for the activation markers CD25 and PD1. In FIG. 4 B, a separate aliquot of cells was stained with 7AAD to identify dead cells. % dead tumor cells=[dead tumor cells (violet⁺7AAD⁺)/total tumor cells (violet⁺)]×100%. Values are the average of triplicates per sample. Error bars indicate standard deviations. Note: The actual ratio of T-cells to tumor cells is less than the indicated ratio since PBMC contain other cells in addition to T-cells.

FIG. 5 (A and B) shows that the anti-CD3/anti-PDL1 fusion protein (BiTE) of the present invention is cytotoxic for human PDL1⁺ human tumor cells and does not kill PDL1⁻ tumor cells. FIG. 5A shows the results of C8161 and MEL1011 human melanoma cells that were stained with antibodies to PDL1 and analyzed by flow cytometry. FIG. 5B shows the results when PBMC from healthy donors were incubated±violet-labeled human PDL1⁺ or PDL1⁻ tumor cells anti-CD3/anti-PDL1 fusion protein as in FIG. 4 and analyzed for % dead tumor cells. PBMC:tumor cell ratio is 20:1. CD8:tumor cell ratio is ˜4:1. Data are from 1 of 3 independent experiments.

FIG. 6 (A, B and C) shows that anti-CD3/anti-PDL1 fusion protein (BiTE) of the present invention activates T-cells and is cytotoxic for chronic myelogenous leukemia (CML) cells. FIG. 6A shows that approximately 20-25% of CIVIL MEG-01 and KU812F cells are PDL1⁺ following treatment with IFNγ. Tumor cells were either untreated (melanomas C8161 and MEL1011) or incubated for 48 hrs with 200 units IFNγ (human CIVIL KU812F and MEG01) and stained with antibody to PDL1. FIG. 6B shows that the anti-CD3/anti-PDL1 fusion protein of the present invention activates CD4⁺ and CD8⁺ T-cells in the presence of PDL1⁺ tumor cells. PBMC incubated with Cell Tracker violet-labeled C8161 cells plus the anti-CD3/anti-PDL1 fusion protein were stained with antibodies to CD3, CD4, CD8, and for activation markers CD25 and CD69. MFI=mean fluorescence intensity. FIG. 6C shows that the anti-CD3/anti-PDL1 fusion protein of the present invention kills ˜25% of the CIVIL cells. Violet-labeled C8161 tumor cells of panel B were analyzed for % dead tumor cells.

FIG. 7 shows that the anti-CD3/anti-PDL1 fusion protein (BiTE) of the present invention activates T-cells that are cytotoxic for PDL1⁺ human melanoma, chronic myelogenous leukemia CML and non-small cell lung cancer cells. PBMC from healthy donors were incubated±violet-labeled human tumor cells ±the anti-CD3/anti-PDL1 fusion protein of the present invention as in FIG. 4 and analyzed for % dead tumor cells. PBMC:tumor cell ratio is 20:1. CD8:tumor cell ratio is ˜4:1. Data are the average+SD of 4, 4, 3, and 2 independent experiments for C8161, MEL1011, MEG-01, KU812F, and H358 cells, respectively. * indicates the PBMC+ anti-CD3/anti-PDL1⁺ tumor value is statistically significantly different from the PBMC+tumor and the anti-CD3/anti-PDL1⁺ tumor values at p<0.05.

FIG. 8 (A and B) shows that the anti-CD3/anti-PDL1 fusion protein (BiTE) activates PBMC from a small cell lung cancer patient that are cytotoxic for PDL1⁺ lung cancer cells. PBMC from a SCLC patient were incubated±violet-labeled human PDL1⁺ (H358) or PDL1⁻ (MEL1011) tumor cells±the anti-CD3/anti-PDL1 fusion protein at a 20:1 ratio of PBMC:tumor cells as in FIG. 4. In FIG. 8A cells were stained with the viability dye 7AAD and the Cell tracker violet stained tumor cells were gated and analyzed for % dead cells. The ratio of CD8 + T-cells to tumor cells is 1.29:1; the ratio of CD4⁺ T-cells to tumor cells is 2.17:1. FIG. 8B shows the percent of CD3⁺ cells that are CD4⁺ or CD8⁺. Separate aliquots of PBMC were labeled with antibodies to CD3, CD4 and CD8, and the CD3 gated cells were analyzed for percent CD3⁺ CD4⁺ and CD3⁺ CD8⁺ cells.

FIG. 9 (A, B and C) shows that the anti-CD3/anti-PDL1 fusion protein (BiTE) activates both CD4⁺ and CD8⁺ cytotoxic T-cells. In FIG. 9A, PBMC from a healthy human donor were either undepleted, or depleted for CD4⁺ T-cells, CD8⁺ T-cells, or CD4⁺+CD8⁺ T-cells. Depleted populations were stained with antibodies to CD3, CD4, and CD8, and the CD3⁺ cells gated and analyzed for CD4 and CD8 expression. Values are the percent of cells in the total PBMC population. In FIG. 9B, violet-labeled human PDL1⁺ (H358) or PDL1⁻ (MEL1011) tumor cells were incubated with the depleted or not depleted PBMC±the anti-CD3/anti-PDL1 and analyzed for percent dead tumor cells as in FIG. 4. PBMC:tumor cell ratio is 20:1. CD8:tumor cell ratio is 5.2:1; CD4:tumor cell ratio is 7.8:1. Data are the average of two experiments. In FIG. 9C, the cells of FIG. 9A were stained with antibodies to CD4, CD8, and CD107a (granule identifying cytotoxic T-cells) and the CD4 and CD3 cells gated and analyzed for CD107a expression. Data are representative of 1 of 2 independent experiments.

FIG. 10 (A and B) FIG. 10 A shows the anti-CD3/anti-PDL1 bispecific fusion protein (BiTE) that binds to both CD3 on a T-cell and binds to PDL1 on a tumor cell. FIG. 10 B shows the gene map for a recombinant construct consisting of VL+VH segments of anti-PDL1 and anti-CD3 antibodies.

FIG. 11 shows that the anti-CD3/anti-PDL1 bispecific fusion protein (BiTE) of the present invention does not induce PDL1 expression on activated T-cells, whereas the OKT3 antibody does induce PDL1 on activated T-cells.

FIG. 12 shows Violet-labeled±C8161 tumor cells were incubated with magnetic bead purified CD3⁺ cells (PBMC) or purified CD3⁺CD56⁺ NKT cells±BiTE (200 ng/mL). Data are representative of three independent experiments. Values with * are significantly different from values without *.

FIG. 13 shows CD3xPDL1 BiTE activates cancer patients' PBMC that are cytotoxic for PDL1+ lung cancer cells. FIG. 13 A. PBMC from a SCLC patient were stained for MDSC (CD11b, CD14, CD15, HLA-DR). CD11b⁺HLA-DR- cells were gated and analyzed by flow cytometry for CD14⁺CD15⁻ M-MDSC and CD15⁺CD14- PMN-MDSC. Data are representative of one of three SCLC patients. FIG. 13 B. SCLC patient's PBMC were labeled with fluorescently-tagged antibodies to CD3, CD4 and CD8, and the gated CD3⁺ cells were analyzed for percent CD3⁺CD4⁺ and CD3⁺CD8⁺ cells. Data are for one of three SCLC patients. FIG. 13C. PBMC from the SCLC patients were incubated±CellTrace violet-labeled PDL1⁺ H358 or PDL1- MEL1011 tumor cells±BiTE at a 20:1 ratio of PBMC:tumor cells. Cells were stained with 7AAD and the CellTrace violet stained tumor cells were gated and analyzed for % dead cells. The ratio of CD8⁺ plus CD4⁺ T cells to tumor cells is 2.3:1. Data are pooled from two independent experiments with PBMC from two individual patients. FIG. 13D. PBMC from a NSCLC patient were stained for MDSC. CD11b⁺HLA-DR- cells were gated and analyzed for CD14⁺CD15⁻ M-MDSC and CD15⁺CD14⁻ PMN-MDSC. FIG. 13E. NSCLC patient's PBMC were labeled with fluorescently-tagged antibodies to CD3, CD4 and CD8, and the CD3 gated cells were analyzed for percent CD3⁺CD4⁺and CD3⁺CD8⁺ cells. FIG. 13 F. PBMC from the NSCLC patient were incubated±CellTrace violet-labeled H358 tumor cells±BiTE at a 20:1 ratio of PBMC:tumor cells. Cells were stained with 7AAD and the CellTrace violet stained tumor cells were gated and analyzed for % dead cells.

FIG. 14 shows CD3xPDL1 BiTE significantly extends the survival time of humanized NSG mice reconstituted with human PBMC and carrying established metastatic human melanoma C8161. NSG mice were inoculated subcutaneously in the right flank with 1×10⁶ human C8161 melanoma cells on day 0. On day 7 when tumors were palpable, mice were either untreated or administered 1×10⁷ human PBMC iv in the tail vein and 0.2 ng (8 ng/kg) CD3xPDL1 BiTE iv in the retro-orbital sinus. On days 8, 9, 10, 11, and 32 mice were given additional iv injections of 0.2 μg BiTE. FIG. 14A. Moribund, euthanized control mouse (tumor+PBMC, no BiTE) showing metastases in the lymph nodes and lungs on day 57. FIG. 14B. Kaplan-Meier plot showing survival. Data are pooled from three independent experiments. FIG. 14C. Representative flow cytometry profiles of splenocytes of moribund/dead BiTE-treated and untreated mice stained for human T cells (CD3, CD4, and CD8 mAbs), or mouse MDSC (Grl and CD11b mAbs). FIG. 14 D. Total numbers and ratio of human T cells and mouse MDSC in the spleens of moribund/dead BiTE-treated and control mice of panel B. Data are pooled from 2-3 mice per group.

FIG. 15 shows CD3xPDL1BiTE does not impact the growth of primary C8161 melanoma in humanized NSG mice. NSG mice were inoculated s.c. in the flank with 1×10⁶ C816 melanoma cells on day 0. Mice were given 1×10⁷ healthy donor PBMC i.v. (tail vein) on day 7 when tumors were palpable, and either untreated or treated with 0.2 ug/mouse/injection for 5 consecutive days staring on day 7. Tumors were measured using a calipers.

### DETAILED DESCRIPTION OF THE INVENTION

Recent clinical trials have demonstrated that a patient's immune system has the capacity to cure cancer patients through T-cell-mediated killing of tumor cells. [1-3] However, only ˜19-30% of patients with limited types of advanced tumors respond to existing immunotherapeutics. Since many solid and hematologic tumors are immune suppressive due to the expression of the co-inhibitory molecule Programmed Death Ligand 1 (PDL1)[4] optimal immunotherapies must simultaneously accomplish three functions: (i) activate cytotoxic CD8⁺ T-cells (CTL); (ii) neutralize PD1 suppression; and (iii) target activated CTL to kill the tumor cells. Existing immunotherapies mediate one or two of these functions, but do not mediate all three simultaneously. The presently claimed bispecific T-cell Engager recombinant protein (anti-CD3/anti-PDL1 (BiTE)) has the potential to simultaneously mediate all three functions. Because most tumors express PDL1, the presently claimed bispecific fusion protein has the potential to treat any type of cancer.

The practice of the present invention will employ, unless otherwise indicated, conventional techniques of immunology, molecular biology, microbiology, cell biology and recombinant DNA, which are within the skill of the art. See, e.g., Sambrook, et al. MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R. I. Freshney, ed. (1987)).

### Definitions

Unless otherwise defined, all technical and scientific terms used herein have the meaning commonly understood by one of ordinary skill in the art to which this invention belongs. The terminology used herein is for the purpose of describing particular embodiments only and is not intended to be limiting of the invention. As used in the description of the invention and the appended claims, the singular forms “a”, “an” and “the” are intended to include the plural forms as well, unless the context clearly indicates otherwise. The following terms have the meanings given:

The term “polynucleotide” as used herein means a sequence of nucleotides connected by phosphodiester linkages. Polynucleotides are presented herein in the direction from the 5′ to the 3′ direction. A polynucleotide of the present invention can be a deoxyribonucleic acid (DNA) molecule or ribonucleic acid (RNA) molecule. Where a polynucleotide is a DNA molecule, that molecule can be a gene or a cDNA molecule. Nucleotide bases are indicated herein by a single letter code: adenine (A), guanine (G), thymine (T), cytosine (C), inosine (I) and uracil (U). A polynucleotide of the present invention can be prepared using standard techniques well known to one of skill in the art.

The term “transfection” of a cell as used herein means that genetic material is introduced into a cell for the purpose of genetically modifying the cell. Transfection can be accomplished by a variety of means known in the art, such as transduction or electroporation.

The term “transgene” is used in a broad sense to mean any heterologous nucleotide sequence incorporated in a vector for expression in a target cell and associated expression control sequences, such as promoters. It is appreciated by those of skill in the art that expression control sequences will be selected based on ability to promote expression of the transgene in the target cell. An example of a transgene is a nucleic acid encoding a bispecific fusion protein of the present invention.

The term “expression vector” as used herein means a vector containing a nucleic acid sequence coding for at least part of a gene product capable of being transcribed. Expression vectors can contain a variety of control sequences, which refer to nucleic acid sequences necessary for the transcription and possibly translation of an operatively linked coding sequence in a particular host organism. In addition to control sequences that govern transcription and translation, vectors and expression vectors may contain nucleic acid sequences that serve other functions as well. The term also includes a recombinant plasmid or virus that comprises a polynucleotide to be delivered into a host cell, either in vitro or in vivo. Preferably the host cell is a transient cell line or a stable cell line and more preferably a mammalian host cell and selected from, but not limited to, the group consisting of HEK293, CHO and NSO or a bacterial cell line, including but not limited to E. coli, or in yeast, including but not limited to saccharomyces cerevisiae.

The term “subject,” as used herein means a human or vertebrate animal including a dog, cat, horse, cow, pig, sheep, goat, chicken, monkey, rat, and mouse.

The term “therapeutically effective amount” as used herein means the amount of the subject compound that will elicit the biological or medical response of a tissue, system, animal or human that is being sought by the researcher, veterinarian, medical doctor or other clinician.

The term “pharmaceutically acceptable” as used herein means the carrier, diluent or excipient must be compatible with the other ingredients of the formulation and not deleterious to the recipient thereof.

The terms “cancer” or “cancerous” as used herein refer to or describe the physiological condition in mammals that is typically characterized by unregulated cell growth. A “tumor” comprises one or more cancerous cells. Examples of cancer include, but are not limited to, carcinoma, lymphoma, blastoma, sarcoma, and leukemia or lymphoid malignancies. More particular examples of such cancers include squamous cell cancer (e.g., epithelial squamous cell cancer), lung cancer including small-cell lung cancer, non-small cell lung cancer (“NSCLC”), adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatome, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, prostate cancer, skin cancer, ocular cancer, brain cancer, lymphoma, leukemia, melanoma as well as head and neck cancer.

The term “recombinant” as used herein means a genetic entity distinct from that generally found in nature. As applied to a polynucleotide or gene, this means that the polynucleotide is the product of various combinations of cloning, restriction and/or ligation steps, and other procedures that result in the production of a construct that is distinct from a polynucleotide found in nature.

The term “peptide,” “polypeptide” and “protein” are used interchangeably to denote a sequence polymer of at least two amino acids covalently linked by an amide bond.

The term “administering” as used herein is defined as the actual physical introduction of the composition into or onto (as appropriate) the host subject. Any and all methods of introducing the composition into the subject are contemplated according to the present invention; the method is not dependent on any particular means of introduction and is not to be so construed. Means of introduction are well-known to those skilled in the art, and preferably, the composition is administered subcutaneously or intratumorally. One skilled in the art will recognize that, although more than one route can be used for administration, a particular route can provide a more immediate and more effective reaction than another route. Local or systemic delivery can be accomplished by administration comprising application or instillation of the immunovaccines into body cavities, inhalation or insufflation of an aerosol, or by parenteral introduction, comprising intramuscular, intravenous, intraportal, intrahepatic, peritoneal, subcutaneous, intracranial, including intracerebral, intrathecal, intratumoral, or intradermal administration.

Although chemotherapeutic agents can induce “immunogenic” tumor cell death and facilitate cross-presentation of antigens by dendritic cells, tumors create a tolerogenic environment that allows them to suppress the activation of innate and adaptive immune responses and evade immunologic attack by immune effector cells. The present invention provides strategies to counteract tumor-induced immune tolerance in the tumor microenvironment and can enhance the antitumor efficacy of chemotherapy by activating and leveraging T-cell-mediated adaptive antitumor immunity against disseminated cancer cells.

The present invention is based on the discovery that the bispecific fusion proteins of the present invention can counteract or reverse immune tolerance of cancer cells. Cancer cells are able to escape elimination by chemotherapeutic agents or tumor-targeted antibodies via specific immunosuppressive mechanisms in the tumor microenvironment and such ability of cancer cells is recognized as immune tolerance. By counteracting tumor-induced immune tolerance, the present invention provides effective compositions and methods for cancer treatment, optional in combination with another existing cancer treatment.

The present invention provides compositions and methods for producing bispecific fusion proteins that counteract immune tolerance in the tumor microenvironment and promote T-cell-mediated adaptive antitumor immunity for maintenance of durable long-term protection against recurrent or disseminated cancers. These bispecific fusion proteins are designed to facilitate effective long term T-cell-mediated immune responses against tumor cells by increasing activation and proliferation of antitumor CD8+ T-cells and CD4+ T-cells by negating immune suppression mediated by regulatory T-cells and myeloid suppressor cells. These antitumor immune responses may be activated in tandem with the sensitization of tumor cells to immune effector-mediated cytotoxicity, thereby establishing a positive feedback loop that augments tumor cytoreduction and reinforces adaptive antitumor immunity.

The bispecific fusion proteins of the invention provide the ability to disrupt immunosuppressive networks in the tumor microenvironment. Tumors employ a wide array of regulatory mechanisms to avoid or suppress the immune response. Cancer cells actively promote immune tolerance in the tumor microenvironment via the expression of cytokines and molecules that inhibit the differentiation and maturation of antigen-presenting dendritic cells (DC). The immunosuppressive cytokines and ligands produced by tumor cells include PDL1 and such increase is correlated with a reduction of survival of patients with diverse types of cancers. The bispecific fusion proteins of the present invention inhibit key immunosuppressive molecules expressed by the targeted tumor.

The present invention provides a method of preventing or treating a neoplastic disease. The method includes administration to a subject in need thereof the bispecific fusion protein of the present invention in combination with another anticancer therapy, wherein the anticancer therapy is a chemotherapeutic molecule, antibody, small molecule kinase inhibitor, hormonal agent, cytotoxic agent, targeted therapeutic agent, anti-angiogenic agent, ionizing radiation, ultraviolet radiation, cryoablation, thermal ablation, adoptive cell therapy, hematopoietic stem cell transplantation or bone marrow transplantation, or radiofrequency ablation.

As used herein, the term “antibody” includes natural or artificial mono- or polyvalent antibodies including, but not limited to, polyclonal, monoclonal, multispecific, human, humanized or fusion antibodies, single chain antibodies, Fab fragments. F(ab′) fragments, fragments produced by a Fab expression library, anti-idiotypic (anti-Id) antibodies (including, e.g., anti-Id antibodies to antibodies of the invention), and epitope-binding fragments of any of the above. The antibody may be from any animal origin including birds and mammals. In one aspect, the antibody is, or derived from, a human, murine (e.g., mouse and rat), donkey, sheep, rabbit, goat, guinea pig, camelid, horse, or chicken. Further, such antibody may be a humanized version of an antibody.

The antibody herein specifically include a “fusion” antibody in which a portion of the heavy and/or light chain is identical with or homologous to corresponding sequences in antibodies derived from one type of antibody, while the remainder of the chain(s) is identical with or homologous to corresponding sequences in antibodies belonging to another antibody type, as well as fragments of such antibodies, so long as they exhibit the desired biological activity.

Various methods have been employed to produce antibodies. Hybridoma technology, which refers to a cloned cell line that produces a single type of antibody, uses the cells of various species, including mice (murine), hamsters, rats, and humans. Another method to prepare an antibody uses genetic engineering including recombinant DNA techniques. For example, antibodies made from these techniques include, among others, fusion antibodies and humanized antibodies. A fusion antibody combines DNA encoding regions from more than one type of species. For example, a fusion antibody may derive the variable region from a mouse and the constant region from a human. A humanized antibody comes predominantly from a human, even though it contains nonhuman portions. Like a fusion antibody, a humanized antibody may contain a completely human constant region. But unlike a fusion antibody, the variable region may be partially derived from a human. The nonhuman, synthetic portions of a humanized antibody often come from Complementarity Determining Regions in murine antibodies. In any event, these regions are crucial to allow the antibody to recognize and bind to a specific antigen.

An antibody fragment can include a portion of an intact, antibody, e.g. including the antigen-binding or variable region thereof. Examples of antibody fragments include Fab, Fab′, F(ab′)2, and Fv fragments; Fc fragments or Fc-fusion products; diabodies; linear antibodies; single-chain antibody molecules; and multi specific antibodies formed from antibody fragment(s). An intact antibody is one which includes an antigen-binding variable region as well as a light chain constant domain (CL) and heavy chain constant domains, CHL CH2 and CH3. The constant domains may be native sequence constant domains (e.g., human native sequence constant domains) or amino acid sequence variant thereof or any other modified Fc (e.g. glycosylation or other engineered Fc).

In the present invention, a linker is used for linking the heavy and light chains of the respective antibodies. In one embodiment, a linker is (GGGGS)n wherein n is 1, 2, 3, 4, 5, 6, 7, or 8. For example, GGGGSGGGGSGGGGS (SEQ ID NO: 12). In another embodiment, a linker is (GGS)n wherein n is 1, 2, 3, 4, 5, 6, 7, or 8. Another linker may include, for example, GGSGGSGGSGGSGGVD (SEQ ID NO: 16). In various aspects, the length of the linker may be modified to optimize binding of the two antibodies to their respective binding sites.

The bispecific fusion proteins of the present invention may be synthesized by conventional techniques known in the art, for example, by chemical synthesis such as solid phase peptide synthesis. Such methods are known to those skilled in the art. In general, these methods employ either solid or solution phase synthesis methods, both well known in the art. Specifically, the methods comprise the sequential addition of one or more amino acids or suitably protected amino acids to a growing peptide chain. Normally, either the amino or carboxyl group of the first amino acid is protected by a suitable protecting group. The protected or derivatized amino acid can then be either attached to an inert solid support or utilized in solution by adding the next amino acid in the sequence having the complementary (amino or carboxyl) group suitably protected, under conditions suitable for forming the amide linkage. The protecting group is then removed from this newly added amino acid residue and the next amino acid (suitably protected) is then added, and so forth. After all the desired amino acids have been linked in the proper sequence, any remaining protecting groups and any solid support are removed either sequentially or concurrently to afford the final polypeptide. By simple modification of this general procedure, it is possible to add more than one amino acid at a time to a growing chain, for example, by coupling (under condition that do not racemize chiral centers) a protected tripeptide with a properly protected dipeptide to form, after deprotection, a pentapeptide.

Typical protecting groups include t-butyloxycarbonyl (Boc), 9-fluorenylmethoxycarbonyl (Fmoc), benxyloxycarbonyl (Cbz), p-toluenesulfonyl (Tos); 2,4-dinitrophenyl, benzyl (Bzl), biphenylisopropyloxy-carboxycarbonyl, cyclohexyl, isopropyl, acetyl, o-nitrophenylsulfonyl, and the like. Of these, Boc and Fmoc are preferred.

Typical solid supports are generally cross-linked polymeric materials. These include divinylbenzene cross-linked styrene-based polymers, for example, divinylbenzene-hydroxymethyl styrene copolymers, divinylbenzene-chloromethyl styrene copolymers, and divinylb enzene-benzhydryl aminopolystyrene copolymers. The divinylbenzene-benzhydrylaminopolystyrene copolymers, as illustrated herein using p-methyl-benzhydrylamine resin, offers the advantage of directly introducing a terminal amide functional group into the peptide chain, which function is retained by the chain when the chain is cleaved from the support.

In one method, the polypeptides are prepared by conventional solid phase chemical synthesis on, for example, an Applied Biosystems, Inc. (ABI) 430A peptide synthesizer using a resin that permits the synthesis of the amide peptide form and using t-Boc amino acid derivatives (Peninsula Laboratories, Inc.) with standard solvents and reagents. Polypeptides containing either L- or D-amino acids may be synthesized in this manner. Polypeptide composition is confirmed by quantitative amino acid analysis and the specific sequence of each peptide may be determined by sequence analysis.

Preferably, the polypeptides can be produced by recombinant DNA techniques by synthesizing DNA encoding the desired polypeptide. Once coding sequences for the desired polypeptides have been synthesized or isolated, they can be cloned into any suitable vector for expression. Numerous cloning vectors are known to those of skill in the art, and the selection of an appropriate cloning vector is a matter of choice. The gene can be placed under the control of a promoter, ribosome binding site (for bacterial expression) and, optionally, an operator (collectively referred to herein as “control” elements), so that the DNA sequence encoding the desired polypeptide is transcribed into RNA in the host cell transformed by a vector containing this expression construction. The coding sequence may or may not contain a signal peptide or leader sequence. Heterologous leader sequences can be added to the coding sequence that causes the secretion of the expressed polypeptide from the host organism. Other regulatory sequences may also be desirable which allow for regulation of expression of the protein sequences relative to the growth of the host cell. Such regulatory sequences are known to those of skill in the art, and examples include those which cause the expression of a gene to be turned on or off in response to a chemical or physical stimulus, including the presence of a regulatory compound. Other types of regulatory elements may also be present in the vector, for example, enhancer sequences.

The control sequences and other regulatory sequences may be ligated to the coding sequence prior to insertion into a vector, such as the cloning vectors described above. Alternatively, the coding sequence can be cloned directly into an expression vector which already contains the control sequences and an appropriate restriction site.

The expression vector may then be used to transform an appropriate host cell. A number of mammalian cell lines are known in the art and include immortalized cell lines available from the American Type Culture Collection (ATCC), such as, but not limited to, Chinese hamster ovary (CHO) cells, HeLa cells, HEK293, baby hamster kidney (BHK) cells, monkey kidney cells (COS), human hepatocellular carcinoma cells (e.g., Hep G2), Madin-Darby bovine kidney (“MDBK”) cells, NOS cells derived from carcinoma cells, such as sarcoma, as well as others. Similarly, bacterial hosts such as E. coli, Bacillus subtilis, and Streptococcus spp., will find use with the present expression constructs. Yeast hosts useful in the present invention include inter alia, Saccharomyces cerevisiae, Candida albicans, Candida maltosa, Hansenula polymorpha, Kluyveromyces fragilis, Kluyveromyces lactis, Pichia guillerimondii, Pichia pastoris, Schizosaccharomyces pombe and Yarrowia lipolytica. Insect cells for use with baculovirus expression vectors include, inter alia, Aedes aegypti, Autographa californica, Bombyx mori, Drosophila melanogaster, Spodoptera frugiperda, and Trichoplusia ni. The bispecific fusion proteins may also be expressed in Trypanosomes.

Depending on the expression system and host selected, the bispecific fusion protein of the present invention is produced by growing host cells transformed by an expression vector described above under conditions whereby the bispecific fusion protein of interest is expressed. The bispecific fusion protein is then isolated from the host cells and purified. If the expression system secretes the bispecific fusion protein into growth media, the bispecific fusion protein can be purified directly from the media. If the bispecific fusion protein is not secreted, it is isolated from cell lysates. The selection of the appropriate growth conditions and recovery methods are within the skill of the art. Once purified, the amino acid sequences of the bispecific fusion protein can be determined, i.e., by repetitive cycles of Edman degradation, followed by amino acid analysis by HPLC. Other methods of amino acid sequencing are also known in the art.

The bispecific fusion proteins of the present invention can be formulated into therapeutic compositions in a variety of dosage forms such as, but not limited to, liquid solutions or suspensions, tablets, pills, powders, suppositories, polymeric microcapsules or microvesicles, liposomes, and injectable or infusible solutions. The preferred form depends upon the mode of administration and the particular cancer type targeted. The compositions also preferably include pharmaceutically acceptable vehicles, carriers or adjuvants, well known in the art, such as human serum albumin, ion exchangers, alumina, and lecithin, buffer substances such as phosphates, glycine, sorbic acid, potassium sorbate, and salts or electrolytes such as protamine sulfate. Suitable vehicles are, for example, water, saline, dextrose, glycerol, ethanol, or the like, and combinations thereof. Actual methods of preparing such compositions are known, or will be apparent, to those skilled in the art. See, e.g., Remington's Pharmaceutical Sciences, Mack Publishing Company, Easton, Pa., 18th edition, 1990.

The above compositions can be administered using conventional modes of delivery including, but not limited to, intravenous, intraperitoneal, oral, intralymphatic, or subcutaneous administration. Local administration to a tumor in question, or to a site of inflammation, e.g., direct injection into an arthritic joint, will also find use with the present invention.

Therapeutically effective doses will be easily determined by one of skill in the art and will depend on the severity and course of the disease, the patient's health and response to treatment, and the judgment of the treating physician.

### Experimental

Below are examples of specific embodiments for carrying out the present invention. The examples are offered for illustrative purposes only, and are not intended to limit the scope of the present invention in any way. Efforts have been made to ensure accuracy with respect to numbers used (e.g., amounts, temperatures, etc.), but some experimental error and deviation should, of course, be allowed for.

### EXAMPLE 1

Production of anti-CD3/anti-PDL1 bispecific fusion protein.

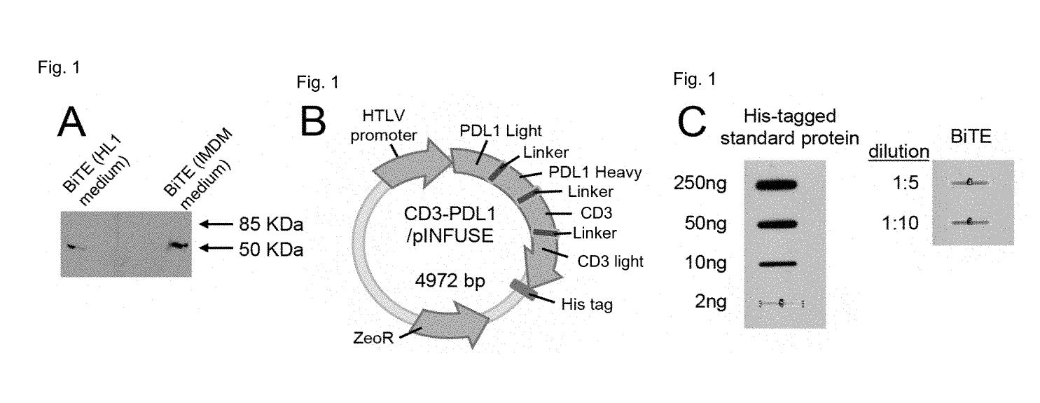

The anti-CD3/anti-PDL1 bispecific fusion protein of the present invention is a ˜55 kDa recombinant protein as assessed by western blotting (FIG. 1A). The anti-CD3/anti-PDL1 bispecific fusion protein was produced in CHO cells transfected with the anti-CD3/anti-PDL1 construct (FIG. 1B). To quantify the production of anti-CD3/anti-PDL1 bispecific fusion protein by the transfectants, CHO-anti-CD3/anti-PDL1 bispecific fusion protein cells were plated at 1×10⁷ cells/100 ml and supernatants were slot blotted and then stained with antibodies to the His tag. Quantification was performed using Image J software as shown in FIG. 1 C.

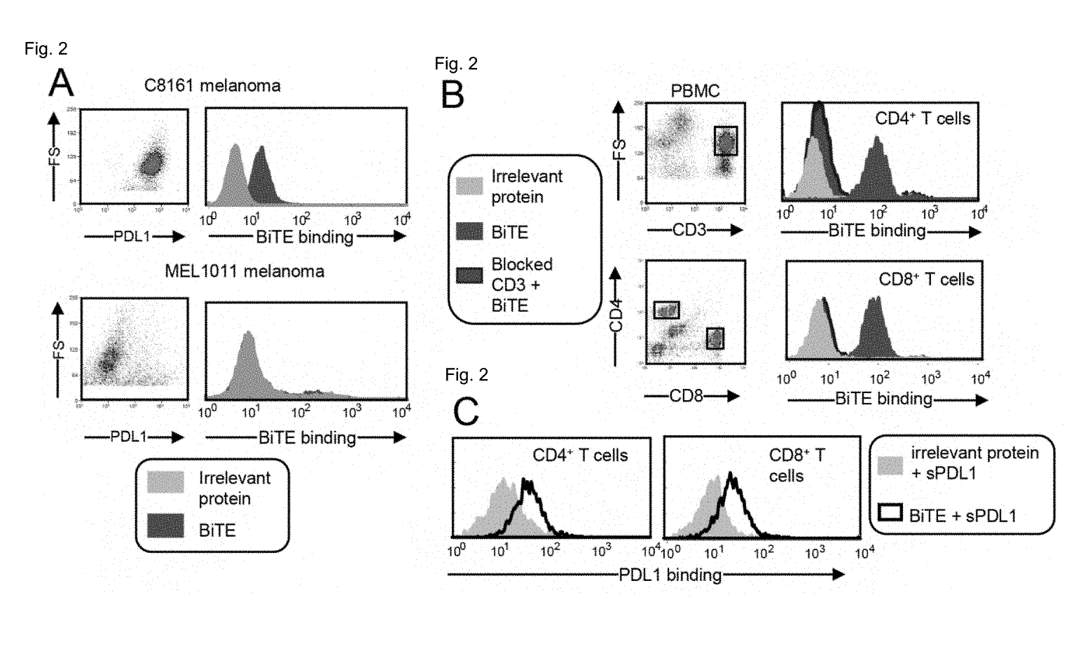

FIG. 2 shows that the anti-CD3/anti-PDL1 bispecific fusion protein binds to PDL1+ human tumor cells and not to PDL1⁻ human tumor cells. Binding activity and specificity were assessed by applying the anti-CD3/anti-PDL1 bispecific fusion protein or an irrelevant recombinant protein (Troy Fc) to PDL1⁺ (C8161) and PDL1⁻ (MEL1011) human tumor cells (FIG. 2A) and to CD3⁺CD4⁺ and CD3⁺CD8⁺ human T-cells (FIG. 2B). Anti-CD3/anti-PDL1 bispecific fusion protein binding was detected using a fluorescently coupled antibody to the His-tag on the anti-CD3/anti-PDL1 bispecific fusion protein. Ability to simultaneously bind PDL1 and CD3 was shown by binding soluble PDL1 (sPDL1) to the anti-CD3/anti-PDL1 bispecific fusion protein to CD3⁺ T-cells as shown in FIG. 2C.

### EXAMPLE 2

Cytotoxicity of 2 Chronic Myelogenous Leukemia (CML) and 2 non-CML cancer cell lines using PBMC from healthy donors and from cancer patients.

To demonstrate that the anti-CD3/anti-PDL1 bispecific fusion protein of the present invention has therapeutic efficacy it was essential to demonstrate (i) the anti-CD3/anti-PDL1 bispecific fusion protein activates T-cells from healthy donors; (ii) activated T-cells kill PDL1⁺ tumor cells and do not have off-target activity; (iii) the anti-CD3/anti-PDL1 bispecific fusion protein activates T-cells that are cytotoxic for multiple PDL1⁺ human tumor cells; (iv) the anti-CD3/anti-PDL1 bispecific fusion protein activates cytotoxic T-cells from cancer patients; and (v) the anti-CD3/anti-PDL1 bispecific fusion protein has the ability to active CD4⁺, CD8⁺ and CD3⁺.

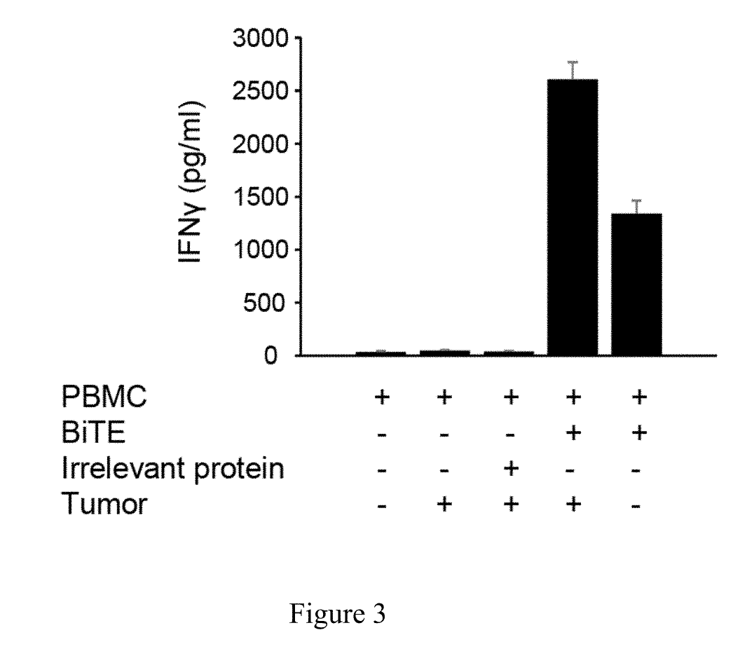

(i) The anti-CD3/anti-PDL1 bispecific fusion protein activates T-cells from healthy donors. Peripheral blood mononuclear cells (PBMC) containing T-cells from healthy human donors were mixed with the anti-CD3/anti-PDL1 bispecific fusion protein (6.5 ng/ml) or an equal amount of an irrelevant control recombinant protein in the presence or absence of PDL1⁺ human melanoma (C8161) cells (FIG. 3). T-cell activation was measured by assessing T-cell production of interferon gamma (IFNγ), a key indicator of T-cell activation. The anti-CD3/anti-PDL1 bispecific fusion protein induced high levels of IFNγ in the PBMC+anti-CD3/anti-PDL1 bispecific fusion protein+tumor cell samples. Significantly lower levels of IFNγ were induced in control PBMC+anti-CD3/anti-PDL1 bispecific fusion protein samples, and essentially no IFNγ was induced in the absence of the anti-CD3/anti-PDL1 bispecific fusion protein, demonstrating that the anti-CD3/anti-PDL1 bispecific fusion protein activates T-cells from healthy donors. This experiment also demonstrated that relatively low concentrations of the anti-CD3/anti-PDL1 bispecific fusion protein (<6.5 ng/ml) are needed to activate T-cells in the presence of tumor.

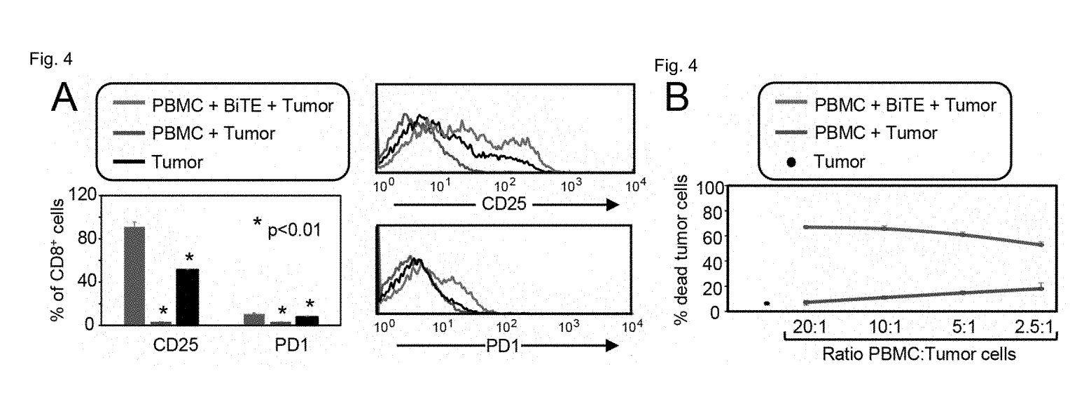

(ii) The anti-CD3/anti-PDL1 bispecific fusion protein-activated T-cells kill PDL1⁺ tumor cells and do not have off-target activity. PBMC from healthy human donors were incubated at 37° C., 5% CO₂ at a 20:1, 10:1, 5:1, or 2.5:1 ratio with Cell Tracker violet-labeled melanoma (C8161) cells in the presence or absence of the anti-CD3/anti-PDL1 bispecific fusion protein (5.2 ng/ml). After 48 hrs. the cells were harvested. Half of the cells were stained with fluorescently-tagged antibodies to CD8, CD25, and PD1; the other half were incubated with the viability dye 7AAD. Flow cytometry analysis revealed that in the presence of tumor, the anti-CD3/anti-PDL1 bispecific fusion protein-activated T-cells as assessed by expression of the activation marker CD25 (alpha chain of the IL-2 receptor). Contrary to other activation strategies, the checkpoint inhibitor receptor PD1 was only minimally expressed as shown in FIG. 4A. Approximately 70% of PDL1⁺ melanoma cells were killed in the presence of the anti-CD3/anti-PDL1 bispecific fusion protein at the 20:1 ratio, and killing titered out to ˜55% at the 2.5:1 ratio. Background levels of killing in the absence of the anti-CD3/anti-PDL1 bispecific fusion protein or PBMC ranged from ˜5%-20% (FIG. 4B).

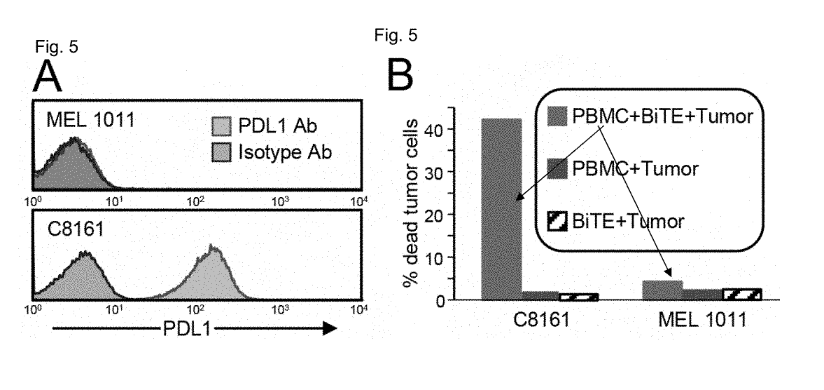

To ascertain that the anti-CD3/anti-PDL1 bispecific fusion protein cytotoxic activity is specific for PDL1⁺ target cells, PDL1- human MEL1011 cells were used as targets (FIG. 5). Less than 5% of MEL1011 cells were killed vs. ˜43% of PDL1⁺ C8161 cells. These results demonstrate that the anti-CD3/anti-PDL1 bispecific fusion protein in conjunction with PBMC efficiently kills PDL1⁺ tumor cells and does not have significant off-target effects on PDL1- tumor cells.

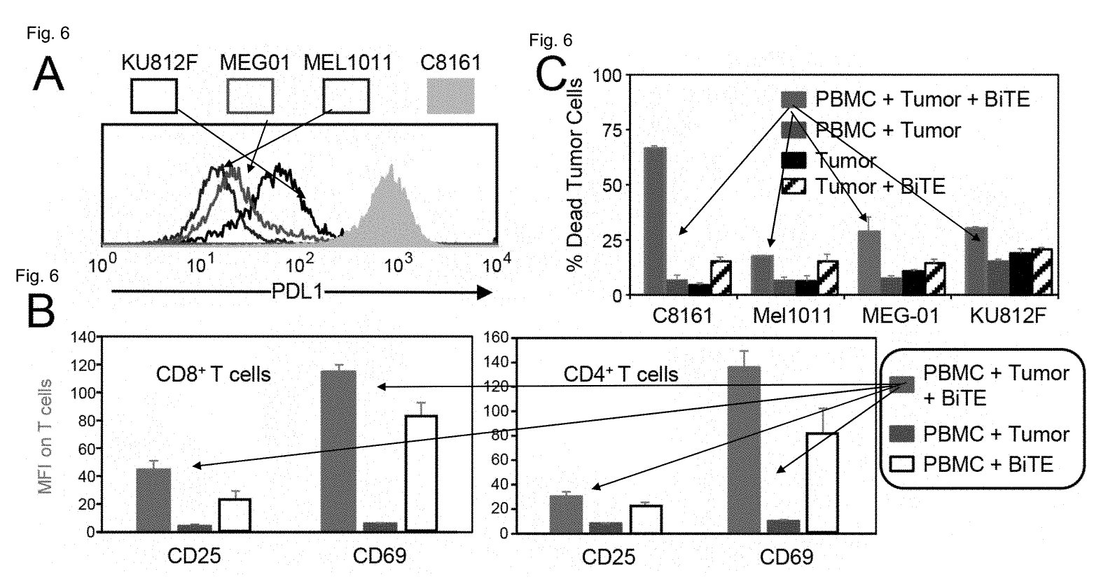

(iii) The anti-CD3/anti-PDL1 bispecific fusion protein activates T-cells that are cytotoxic for multiple PDL1⁺ human tumor cells. To determine if the anti-CD3/anti-PDL1 bispecific fusion protein is globally effective against PDL1⁺ tumors, two lines of chronic myelogenous leukemia (CML) were tested as target tumor cells. The MEG-01 and KU812F CML cell lines contain ˜21% and 30% PDL1+ cells (FIG. 6A). Therefore, anti-CD3/anti-PDL1 bispecific fusion protein-mediated cytotoxicity would be expected to be in the range of 20%-30%. Using the same procedure as described for FIG. 4, anti-CD3/anti-PDL1 bispecific fusion protein activated T-cells, as assessed by expression of the T-cell activation markers CD25 and CD69 (FIG. 6B), and killed ˜25-30% of MEG-01 and KU812F cells (FIG. 6C).

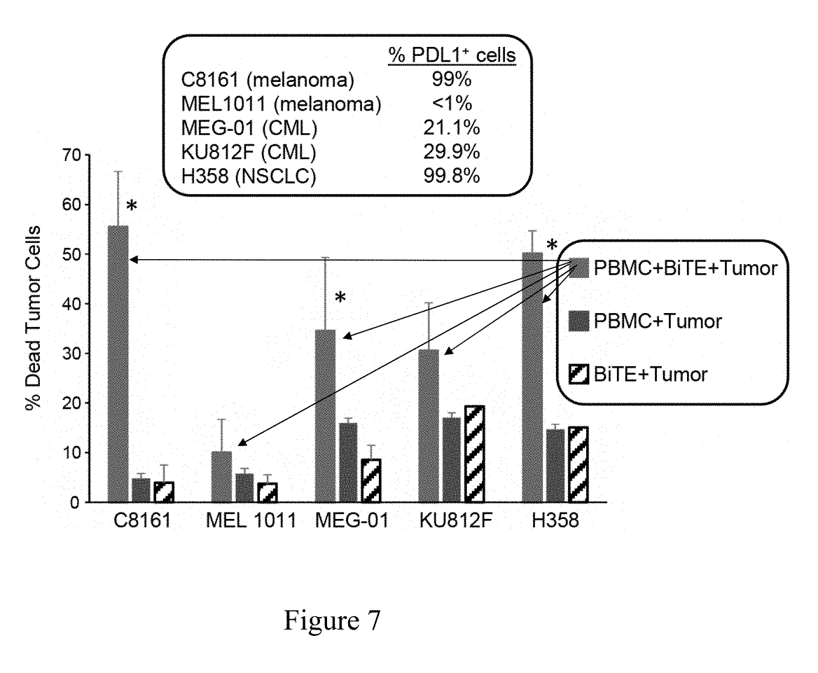

To further confirm that the anti-CD3/anti-PDL1 bispecific fusion protein of the present invention targets PDL1⁺ tumor cells regardless of the type of tumor, a lung adenocarcinoma line, H358, which has >99% PDL1⁺ cells was tested and exhibited >50% tumor cell death (FIG. 7). These studies demonstrate that the anti-CD3/anti-PDL1 bispecific fusion protein of the present invention activates T-cells from healthy donors that are cytotoxic for PDL1⁺ CML, melanoma, and lung cancer cells.

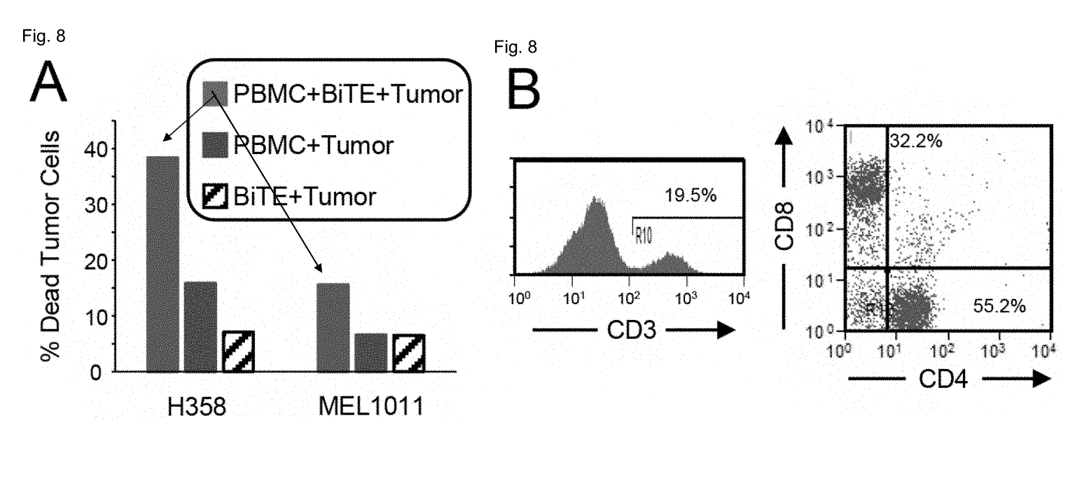

(iv) The anti-CD3/anti-PDL1 bispecific fusion protein of the present invention activates T-cells from cancer patients that are cytotoxic for PDL1⁺ tumor cells. Since the anti-CD3/anti-PDL1 bispecific fusion protein of the present invention has the potential to be efficacious in any cancer patient with PDL1⁺ tumor cells, PBMC from a patient with small cell lung cancer (SCLC) were tested. Using the cytotoxicity assay described in FIG. 4, PDL1⁺ H358 lung cancer cells and PDL1-MEL1011 cells were Cell Tracker violet labeled and incubated with PBMC+anti-CD3/anti-PDL1 bispecific fusion protein of the present invention, PBMC, or the anti-CD3/anti-PDL1 bispecific fusion protein. Approximately 38% of the PDL1⁺ C8161 cells vs. ˜15% of the PDL1-MEL1011 melanoma cells were killed when incubated with PBMC plus the anti-CD3/anti-PDL1 bispecific fusion protein of the present invention as shown in FIG. 8A. To determine the actual T-cell to tumor cell ratio, the cells from panel A were labeled with fluorescently-tagged antibodies to CD3, CD4 and CD8, and the percent of CD3⁺CD4⁺ and CD3⁺CD8⁺ T-cells assessed (FIG. 8B). Although the PBMC to tumor ratio was 20:1, the actual CD8 to tumor ratio was 1.29:1 and the CD4 to T-cell ratio was 2.17:1. These results demonstrate that the anti-CD3/anti-PDL1 bispecific fusion protein of the present invention activates T-cells from cancer patients and that ratios less than three (3) T-cells per tumor cell are needed for killing.

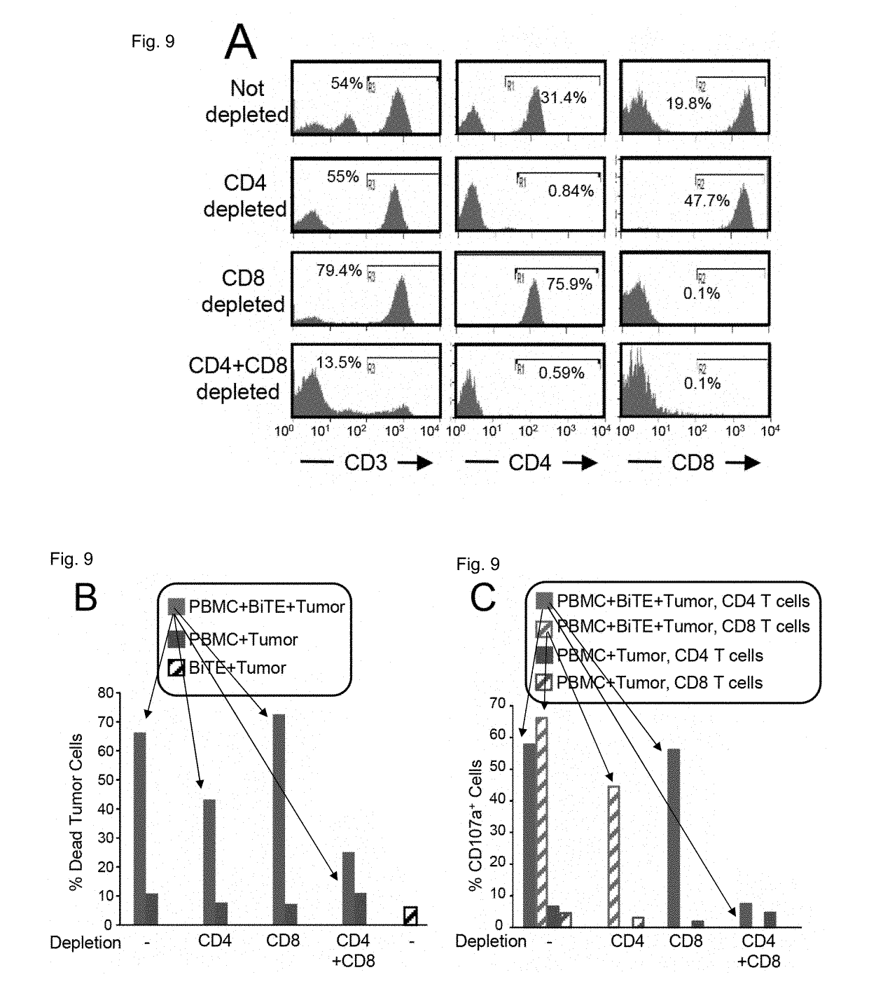

(v) The anti-CD3/anti-PDL1 bispecific fusion protein of the present invention mediates tumor cell cytotoxicity by activating both CD4⁺ and CD8⁺ T-cells, and it is theorized also CD3⁺ NKT cells. Optimal T-cell-based immunotherapy should activate the maximum number of effector cells. CD8⁺ T-cells and natural killer (NK) cells are traditionally considered the optimal effector cells. Since hypothetically the anti-CD3/anti-PDL1 bispecific fusion protein of the present invention should activate any CD3⁺ cell, it has the potential to activate CD3⁺CD8⁺ and CD3⁺CD4⁺ T-cells, as well as CD3⁺ NKT-cells. To determine which cells are activated and have cytotoxic activity, PBMC were depleted for CD4⁺, CD8⁺, or CD4⁺ plus CD8⁺ cells and subsequently tested for cytotoxic activity in the presence of the anti-CD3/anti-PDL1 bispecific fusion protein of the present invention and C8161 melanoma cells. Depleted populations contained <1% of the depleted cells (FIG. 9A). Simultaneous depletion of CD4⁺ plus CD8⁺ T-cells reduced killing by 61% and depletion of CD4+ cells by 34% (FIG. 9B). Killing was not reduced by depletion of CD8+ cells. Since depletion of one cell populations results in increased numbers of the other populations, these results indicate that both additional CD3⁺ non-CD8⁺ or -CD4⁺ cells are rendered cytotoxic by the anti-CD3/anti-PDL1 bispecific fusion protein of the present invention. Since NKT-cells have known anti-tumor cytotoxic activity and are CD3⁺, they may be another population activated by the anti-CD3/anti-PDL1 bispecific fusion protein of the present invention. The cells of panel B were further stained with fluorescently-tagged antibodies to CD3, CD4, CD8, and the marker CD107a which identifies cells containing granules releasing the lytic molecule perforin which is responsible for target cell lysis (FIG. 9C). Gated CD3⁺CD4⁺ and CD3⁺CD8⁺ populations contained >45% CD107a⁺ cells. These results further confirm that the anti-CD3/anti-PDL1 bispecific fusion protein of the present invention renders both CD4⁺ and CD8⁺ T-cells cytotoxic for PDL1⁺ targets.

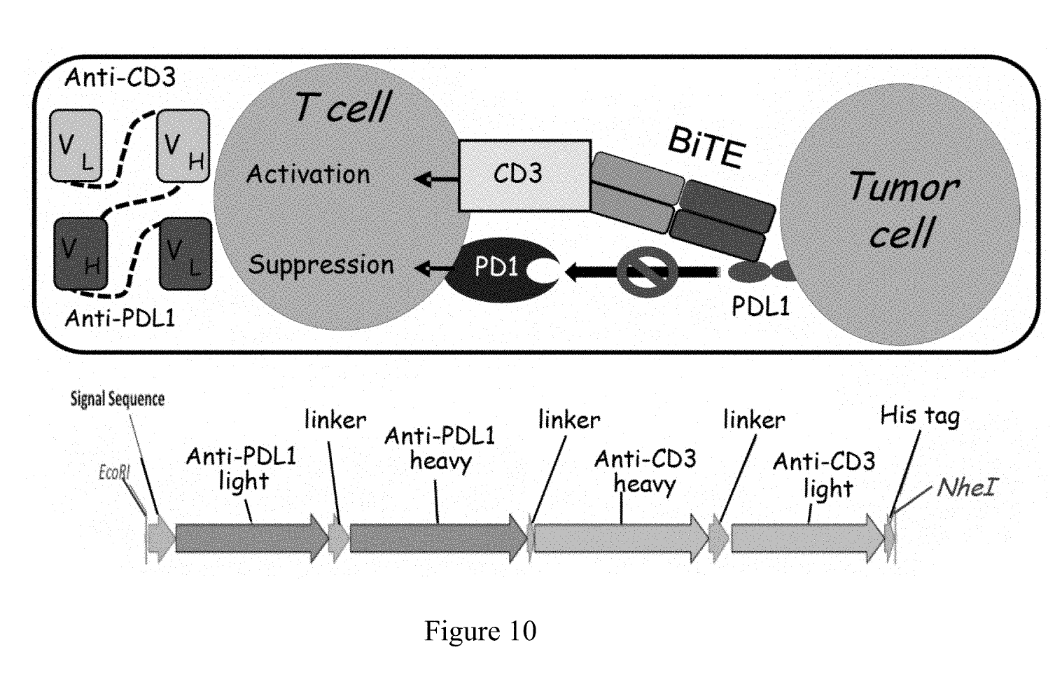

The anti-CD3/anti-PDL1 bispecific fusion protein of the present invention comprises the variable regions of the heavy (VH) and light (VL) chains of antibodies that bind to the CD3 component of the T-cell antigen receptor and to PDL1 as shown in FIG. 10. This novel recombinant protein allows for activation of T-cells through CD3, while simultaneously redirecting T-cells to PDL1⁺ tumor cells, neutralizing PD1-mediated suppression, and killing the tumor cells. The anti-CD3 sequences from the OKT3 clone[5] and the anti-PDL1 sequences from the 12A4 clone[6] were used in the fusion protein since the CD3 sequence has shown efficacy in other fusion proteins[7] and PDL1 antibody has shown efficacy as a monotherapy[8-9].

To function, the anti-CD3/anti-PDL1 bispecific fusion protein must bind to CD3 on T-cells and PDL1 on tumor cells. Binding activity was confirmed by comparing the binding of a fluorescently tagged anti-BiTE antibody by PDL1⁺ human melanoma and CD3⁺ human T-cells either incubated with or without the anti-CD3/anti-PDL1 bispecific fusion protein.

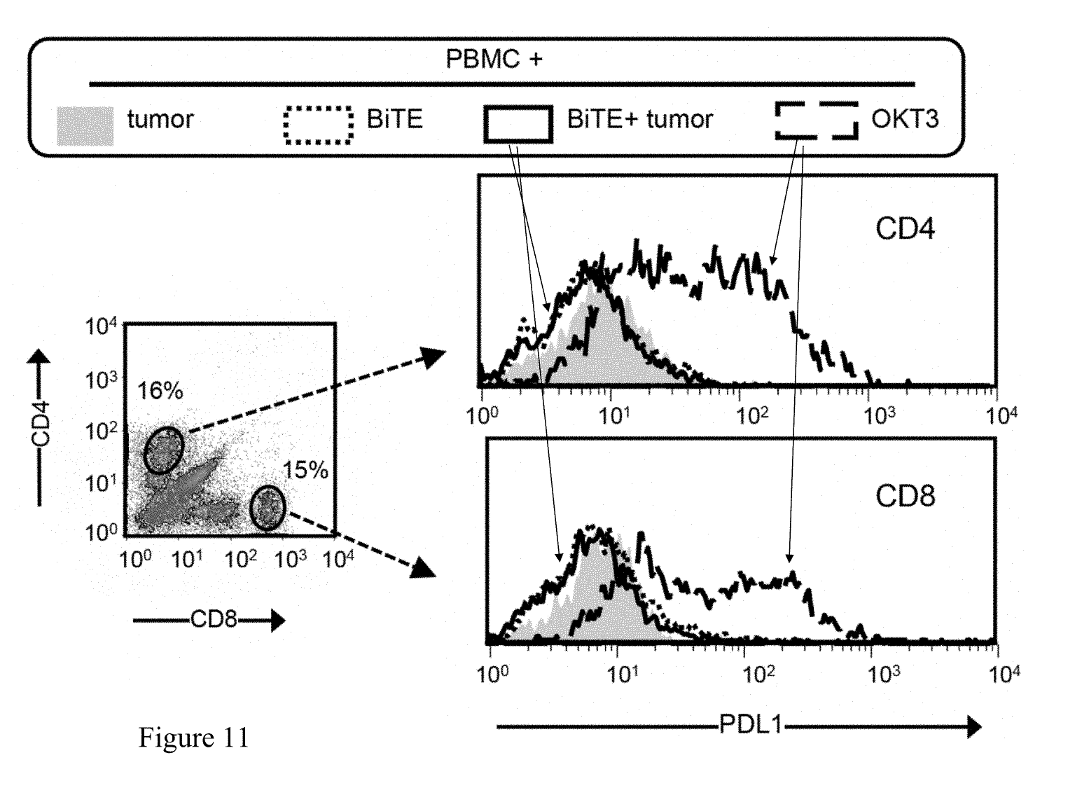

FIG. 11 shows that in contrast to the OKT3 antibody, the anti-CD3/anti-PDL1 bispecific fusion protein does not induce PDL1 expression on activated T-cells. PBMC from healthy human donors were incubated with PDL1+ human melanoma cells (C8161) in the presence or absence of the anti-CD3/anti-PDL1 bispecific fusion protein (0.12 pg/mL) or with OKT3 (anti-CD3) mAb (10 pg/mL). At the end of the 48 hr incubation, cells were harvested and stained for CD4, CD8, and PDL1. Gated CD4+ and CD8+ cells were analyzed for PDL1.

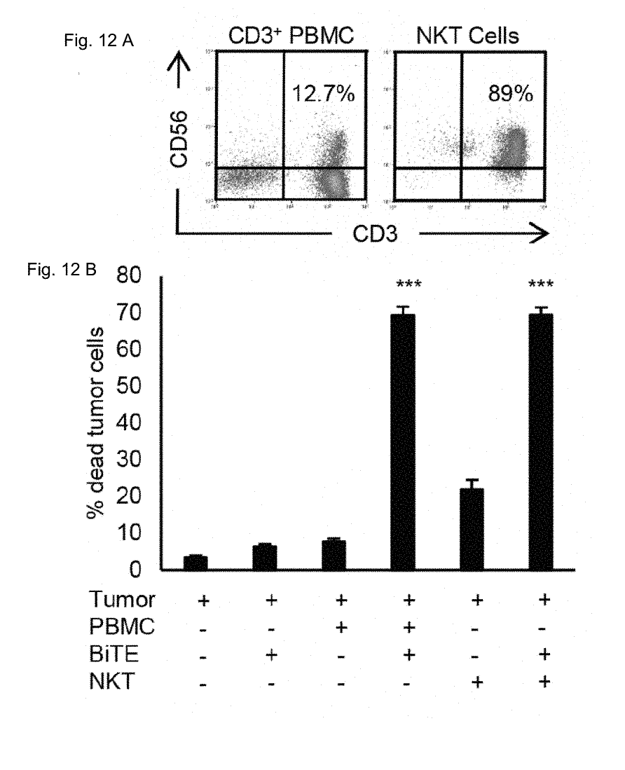

Simultaneous depletion of CD4⁺ and CD8⁺ T cells did not eliminate cytotoxicity, suggesting that other CD3⁺ cells in the PBMC might also be activated by the BiTE. Since NKT cells are CD3⁺, CD3⁺CD56⁺ NKT cells were purified and assessed their ability to kill PDL1⁺ C8161 human melanoma cells in the presence or absence of BiTE (FIG. 12). To be consistent with earlier experiments, all samples were tested at a 20:1 ratio of PBMC to tumor cells. In the presence of BiTE, NKT cells were just as cytotoxic for C8161 cells as total CD3⁺ PBMC. These results confirm that the BiTE renders both CD4⁺ and CD8⁺ T cells and NKT cells cytotoxic for PDL1⁺ target cells.

### EXAMPLE 3

The CD3xPDL1 BiTE Activates Cytotoxic PBMC From Cancer Patients

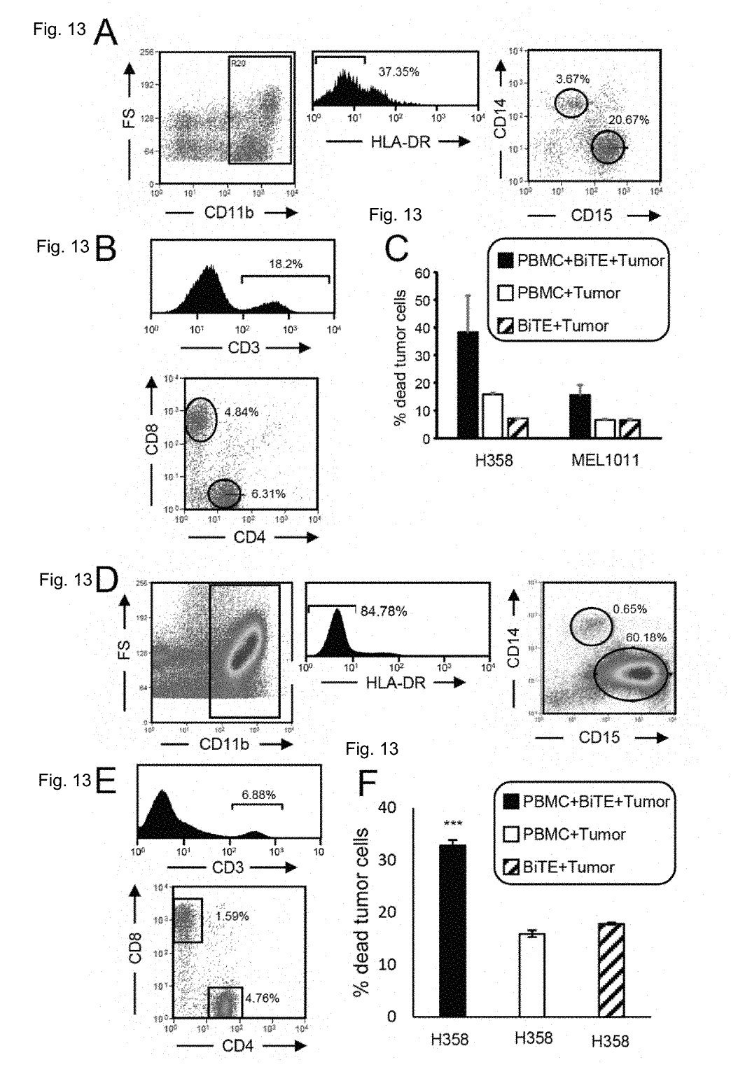

Since the goal of these studies is to determine if the CD3xPDL1 BiTE will be efficacious in cancer patients, PBMC from patients with small cell lung cancer (SCLC) and non-small cell lung cancer (NSCLC) were tested. MDSC are frequently present in the PBMC of SCLC and NSCLC patients [10, 11] and could reduce the function of the CD3xPDL1 BiTE. Using the accepted markers for identifying human MDSC [12], PBMC from three SCLC patients were screened by flow cytometry for MDSC. The leukocytes of these patients contained an average of 37.0%±11.9 total MDSC, 1.5%±1.4 monocytic MDSC (M-MDSC), and 35.5%±12.8 granulocytic MDSC (PMN-MDSC). A representative profile of a SCLC patient's PBMC stained for MDSC is shown in FIG. 13A. The percent of PBMC that were CD3⁺CD4⁺ and CD3+CD8+ T cells was also determined (FIG. 13B). To determine if the patients' PBMC could be activated by the BiTE, H358 lung adenocarcinoma cells were labeled with CellTrace Violet and incubated with SCLC patient PBMC with or without BiTE. Approximately 38% of the PDL1⁺ H358 cells vs. ˜15% of the PDL1- MEL1011 melanoma cells were killed when incubated with PBMC plus BiTE (FIG. 13C). The PBMC to tumor ratio was 20:1 in the cytotoxicity assay; however, since CD8⁺ plus CD4⁺ T cells are only 11.15% of the PBMC, the actual T cell to tumor cell ratio was 2.3:1.

PBMC from a non-small cell lung cancer (NSCLC) patient were also tested. Approximately 60% of this patient's leukocytes were PMN-MDSC, 1.4% were M-MDSC (FIG. 13D), 4.76% were CD3⁺CD4⁺, and 1.59% were CD3+CD8+ (FIG. 13E). Similar to the SCLC patient, BiTE treatment of the NSCLC patient's PBMC also resulted in significant cytotoxicity of PDL1⁺ C8161 tumor cells (FIG. 13F). These results demonstrate that the BiTE activates T cells from cancer patients despite the presence of MDSC, and that ratios less than four T cells per tumor cell are needed for killing.

### EXAMPLE 4

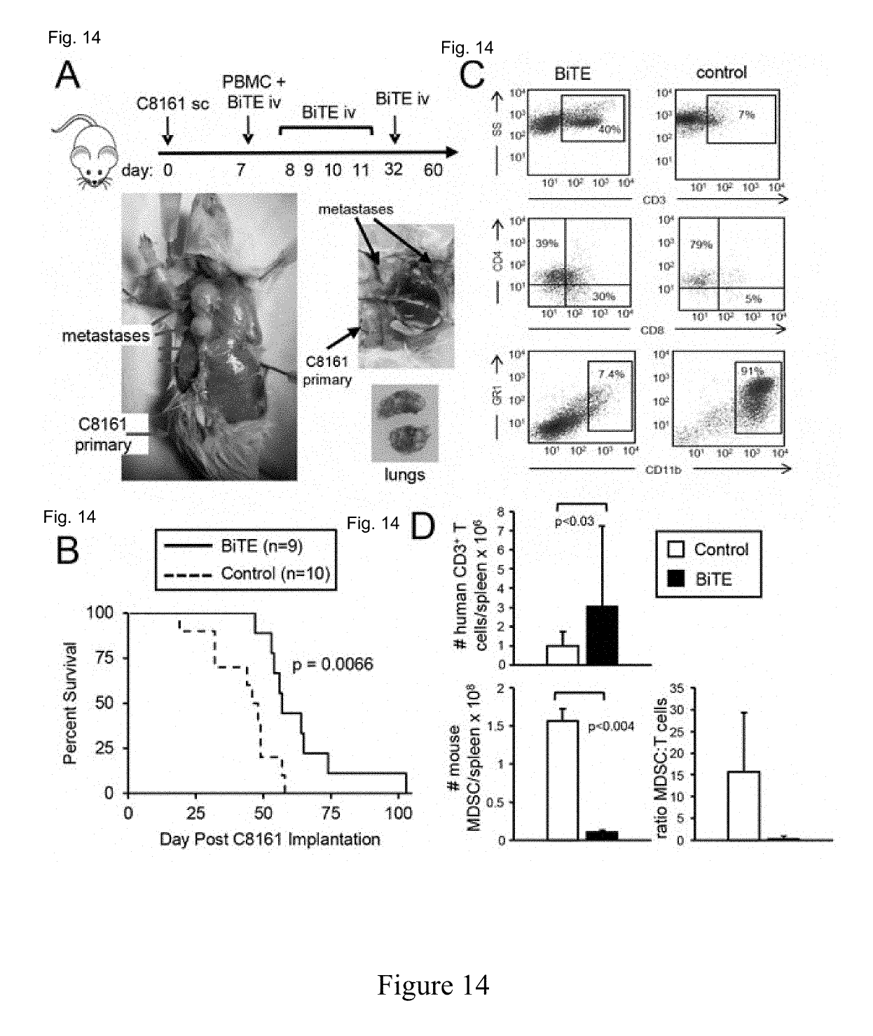

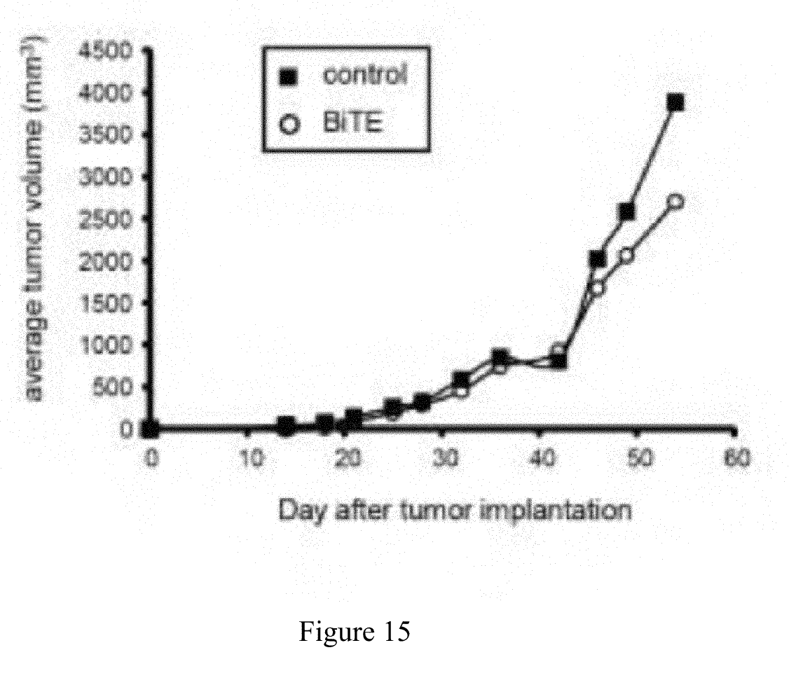

CD3xPDL1 BiTE significantly extends the survival time of humanized tumor-bearing NSG mice To determine if the CD3xPDL1 BiTE has in vivo efficacy, we have used humanized NSG mice [13]. NSG mice are immune deficient and are readily engrafted by human PBMC and human tumor cells, and therefore serve as a model for studying the in vivo effects of the human immune system on human tumors. NSG mice were inoculated s.c. in the right rear flank with 1×10⁶ PDL1⁺ human C8161 melanoma cells. When tumors were palpable (day 7), mice were reconstituted with healthy donor human PBMC. Treated mice received five daily injections of BiTE starting on day seven. Control mice did not receive BiTE (FIG. 14A). Engrafted mice did not display symptoms of graft-vs.-host disease or symptoms of autoimmunity during the course of the experiments. C8161 is spontaneously metastatic in the reconstituted NSG mice as evidenced by metastatic disease in multiple sites including lymph nodes and lungs. Mean survival time±SE of BiTE-treated mice was significantly longer than MST of control mice (63.66±5.58 vs. 43.3±3.86 days, respectively; p=0.005) (FIG. 14 B). A subset of these mice were also followed for growth of primary tumor. There was no difference in progression of primary tumor between the BiTE and control-treated groups (FIG. 15). The spleens of this latter subset were analyzed at day 54 (42 days after the last BiTE treatment and 47 days after transfer of the PBMC) for their content of CD3⁺, CD4⁺, and CD8⁺ T cells. Representative profiles are shown in FIG. 14C, and values are quantified in FIG. 14D. The spleens of the control-treated mice were much larger and contained 10 times as many cells as the spleens of BiTE-treated mice (1.6×10⁸ vs. 1.6×10⁷ cells, respectively). Since such spleen enlargement is characteristic of the accumulation of MDSC, splenocytes of the mice were stained for phenotypic markers of both human and mouse MDSC (FIG. 14C). Human MDSC were not present however, ˜90% of the splenocytes in the control mice were phenotypically mouse MDSC (Gr1⁺CD11b⁺ cells), while the spleens of BiTE-treated mice contained many fewer mouse MDSC. Although the overall number of splenic CD3⁺CD4⁺ and CD3⁺CD8⁺ T cells in control and BiTE-treated mice was similar, the ratio of MDSC to T cells in control mice was significantly greater than the ratio in BiTE-treated mice. These results demonstrate that the CD3xPDL1 BiTE extends the survival time of humanized mice with established, spontaneously metastatic PDL1⁺ tumor cells.

### Discussion

The studies described here demonstrate that the CD3xPDL1 BiTE binds simultaneously to T cell-expressed CD3 and tumor cell-expressed PDL1. In conjunction with PBMC, the CD3xPDL1 BiTE facilitates the specific lysis of PDL1⁺ tumor cells by activating both CD4⁺ and CD8⁺ cytotoxic T cells and NKT cells. In vivo studies demonstrated that a short treatment with the CD3xPDL1 BiTE significantly extended the survival time of humanized NSG mice carrying an established metastatic human melanoma. The rational for generating this particular BiTE was that it would serve the dual purpose of activating anti-tumor immunity while concurrently minimizing PD1-mediated immune suppression.

Binding of the CD3xPDL1 BiTE to CD3⁺ and PDL1⁺ cells is roughly linearly proportional to the amount of CD3 or PDL1 expressed by the target cells. In contrast, PBMC-mediated BiTE killing of target cells does not require high levels of PDL1 since low levels of BiTE binding facilitate similar levels of target cell killing as higher levels of BiTE binding. Although some tumor cells constitutively express PDL1, other tumor cells only express PDL1 after induction by IFNγ. Such induction occurs in the tumor microenvironment when solid tumors are infiltrated by activated IFNγ-secreting T cells [14]. Therefore, the CD3xPDL1 BiTE may be effective in activating antitumor immunity against tumor cells that constitutively express low levels of PDL1 as well as in situations where there is already an active T cell-mediated antitumor immune response and IFNγ is driving the expression of PDL1.

The studies with PBMC from lung cancer patients demonstrated that T cells activated by the CD3xPDL1 BiTE are not inhibited by MDSC, suggesting that patients with high levels of MDSC may be candidates for BiTE therapy. Resistance to MDSC could have occurred because some MDSC express PDL1 and the BiTE mediates lysis of these cells. In addition to MDSC, there are other immune suppressive mechanisms that are active in individuals with cancer and which have the potential to interfere with BiTE function. BiTEs are known to convert existing Tregs to T cytotoxic cells [15]. In the case of the CD3xEGFRvIII BiTE, the conversion of Tregs to cytotoxic effector cells occurred by induction of granzyme and perforin in the Tregs [16]. Therefore, as for other BiTEs, the CD3xPDL1 BiTE may be effective in individuals with high levels of Tregs. Whether BiTE-activated T cells are protected against immune suppressive soluble molecules such as indole-amine 2,3-dioxygenase (IDO) [17], TGFβ [18], or other soluble molecules is not known. In addition to immune suppression by MDSC and Treg-specific mechanisms, tumor-associated macrophages, fibroblasts, mast cells, B cells, and T cells themselves within solid tumors may become PDL1⁺ in response to IFNγ [14]. In vitro studies using a combination of a CD3xCD33 BiTE with anti-PDL1 antibodies demonstrated that blockade of the PD1/PDL1 axis augmented BiTE-mediated killing of AML cells, demonstrating that BiTE activation does not by itself overcome PD1-mediated suppression [19]. Since the CD3xPDL1 BiTE targets PDL1+ cells, it may also minimize PD1 pathway immune suppression that is likely to escalate as T cell anti-tumor immunity and levels of IFNγ increase in the tumor microenvironment.

However, activated T cells may also express PDL1, so the CD3xPDL1 BiTE may also lyse some of these cells. This activity could limit the effector cell repertoire and thereby counter-act antitumor immunity. However, this potential “off-tumor” effect has not prevented the BiTE from significantly extending survival time in the humanized mouse studies described here.

Human myeloid cell subsets differentiate and accumulate in NSG mice reconstituted with human hematopoietic stem cells [20]; however, the PBMC-reconstituted NSG mice in the current study contained only very low levels of human MDSC. In contrast, the control humanized tumor-bearing NSG mice contained very high levels of mouse MDSC. This finding is not unexpected since the mutations in NSG mice do not affected mouse myeloid cell differentiation [21]. What is surprising, however, is that the BiTE-treated mice have many fewer mouse MDSC and a much lower ratio of mouse MDSC to human T cells. MDSC levels are typically regulated by tumor burden since MDSC are induced by tumor-secreted factors [22]. However, the difference in MDSC levels between control and BiTE-treated mice is not due to differences in the amount of tumor since MDSC levels were determined when both groups were moribund and had similar tumor burdens. The CD3xPDL1 BiTE does not react with mouse PDL1⁺ cells, so the BiTE's effect is not a direct killing of PDL1⁺ mouse MDSC. The CD3xPDL1 BiTE could be down-regulating tumor-induced inflammation and thereby diminishing the accumulation of MDSC since inflammation is a major driving force for MDSC [23]. Whether the relative reduction in MDSC in the BiTE-treated mice contributes to the extended MST is unclear since it is not known if mouse MDSC cross-species barriers and suppress human T cells.

Although the BiTE-treated humanized mice had a significantly extended survival time, they eventually died from metastatic disease. Extensive work has demonstrated that BiTEs have a very short half-life in vivo and that therapeutic efficacy requires continual infusion [24]. The CD3xPDL1 BiTE was administered once a day for five days starting when tumors were palpable (Day 7 after transplant), and a sixth treatment on day 32. Therefore, BiTE was not present during later stages of tumor progression. Given the kinetics of tumor growth and metastasis, it is likely that this scheduling prolonged survival because it delayed or perturbed the metastasis process.

BiTEs have shown efficacy in the clinic and in general are well tolerated. However, clinical responses in some patients are accompanied by significant adverse effects. In three clinical trials, some of the acute lymphoblastic leukemia patients treated with Blincyto (CD3xCD19 BiTE) experienced central nervous system or cytokine release syndrome (CRS) adverse events [25]. Less severe CRS can be treated with the glucocorticoid dexamethasone [25] which is most likely effective because it decreases cytokine production but does not inhibit BiTE-mediated cytotoxic activity [26]. We observed no adverse effects in the NSG-treated mice; however, such effects rarely occur and are difficult to identify in mice. Studies with cancer patients will be necessary to definitively determine the safety of the CD3xPDL1 BiTE.

The studies reported here suggest that the CD3xPDL1 BiTE is a novel reagent that because of its dual ability to activate tumor-reactive T cells and NKT cells and direct them to PDL1⁺ target cells, may be a useful therapeutic. Since not all tumor cells are, or become, PDL1⁺, the CD3xPDL1 BiTE can be useful in combination with other immunotherapies that have modest immune cell activation activity and do not have the capability of neutralizing PD1-mediated immune suppression.

### Materials and Methods

### Construct Design and Generation of Recombinant Protein

VL, VH regions of the human 12A4 anti-human PDL1 monoclonal antibody [6] and VH and VL regions of the OKT3 anti-human CD3 monoclonal antibody [27] were linearly assembled including 15 base linkers between each segment and a 6 residue his tag at the 3′ end. The OKT3 antibody was originally a mouse mAb but has been humanized [27]. EcoR1 and Nhel restriction sites at the 5′ and 3′ ends, respectively, served as sites for cloning the BiTE into the pINFUSE-hIgG1-Fc1 vector. The construct was transformed into DH5a cells and transformed DH5a cells were selected using 25 μg/ml zeocin (Invitrogen). The BiTE construct was transfected into CHO cells by Amaxa electroporation [11] and transfectants were selected using zeocin (400 ug/ml; Invitrogen). A high expressing BiTE/CHO clone (H4) was obtained by limiting dilution cloning. Transfectants were expanded in IMDM supplemented with 1% AA, 1% β-mercaptoethanol, 1% Glutamax, 0.1% Gentamicin and 400 ug/mL Zeocin. To obtain BiTE for experiments, transfectants were plated at 3×107 cells/30 mL of serum-free HL1 medium (Lonza, Biowhittaker) supplemented with 1% AA, 1% Glutamax, and 0.1% Gentamicin and cultured for 2-4 days at 37° C. Supernatants were then collected, centrifuged to remove cellular debris, and 120 ml aliquots were concentrated 20 fold to 6 mL using Ultra-15 10 kD Centrifugal Filter Units (Millipore/Amicon) to eliminate molecules >10 KD. BiTE was identified by western blotting with anti-6xhis mAb (AD1.1.10, Invitrogen), and quantified by comparing band density of slot-blotted BiTE to a his-tagged standard protein (CD80-Fc, R&D) using ImageJ software. BiTE was stored at 40^{C} until used. Concentrated supernatant from BiTE-transfected CHO cells and similarly concentrated supernatant from non-BiTE transfected CHO cells were electrophoresced on denaturing 12% SDS-PAGE gels, and the resulting gels were stained with Coomassie blue dye to assess purity. BiTE supernatants contained a prominent band at 55 kD and a less prominent band of unknown identification at ˜70 kD. The ˜70 kD band, but not the ˜55 kD band, was also present in the non-BiTE supernatants.

Cells, Antibodies, Control Recombinant Proteins, Healthy Donor and Patient PBMC CHO, CML lines MEGO1 [29] and KU812F [30] cells were obtained from the ATCC. CHO cells were cultured in IMDM medium supplemented with 10% FCS, 1% Glutamax, 1% gentamycin, 1% penicillin, and 1% streptomycin. KU812F and MEGO1 cells were cultured in RPMI 1640 supplemented with 10% FCS, 1% Glutamax, 1% gentamycin, 1% penicillin, and 1% streptomycin or serum-free HL1 medium supplemented as per IMDM medium. Jurkat cells were cultured in IMDM supplemented with 10%FCS, 1% penicillin-streptomycin, 1% Glutamax, 0.1% gentamycin, and 5×10-5M β-mercaptoethanol. Characterization and culturing of human lung adenocarcinoma H358, and melanomas C8161 [30] and 1011 were previously described [11, 31, 32].

Antibodies to human PDL1 (CD274; clone 29E.2A3; BV421 and PE-Cy7), PD1 (clone EH12.2H7, PE-Cy7), CD3 (clone OKT3, FITC, PE, APC), CD3 (clone UCHT1, BV421), CD4 (clone OKT4, PB, BV510, APC, APC-Cy7, PE-Cy7), CD8 (clone SK1, APC), CD8a (clone HIT8a, PE), CD69 (clone FN50, AlexaF488), HLA-DR (clone L243, BV421), CD14 (clone HCD14, FITC), CD15 (clone HI98, PE), CD1lb (clone ICRF44, APC), CD56 (clone HCD56, FITC), LEAF purified anti-human CD3 (clone OKT3), LEAF-purified anti-human PDL1 (clone 29E.2A3) were from BioLegend. Antibodies to human PD1 (clone MIH4, PE), PDL1 (clone MIH1, PE), CD25 (clone M-A251, APC-Cy7), CD80 (clone L307.4, PE), CD80 (clone L307.4, FITC), and CD25 (clone M-A251, PE-Cy7, APC-Cy7) were from BD Pharmingen. Antibodies to human PDL1 (clone MIH1, PE-Cy7), CD4 (clone OKT4, FITC, eFluor605), CD8 (clone OKT8, FITC), and CD8a (clone IT8a, PE) were from eBioscience. Control recombinant protein TROY-Fc was from R&D. Antibodies to mouse CD1lb (clone M1/70, APC) and mouse Grl (clone Rb6.8C5, PacB) were from BioLegend.

Human PBMC from healthy donors were prepared on ficoll gradients and stored in liquid nitrogen until used as previously described [11]. PBMC used for NKT purification were obtained from AllCells, LLC (Alameda, CA) and were handled according to the manufacturer's direction. Fresh samples of blood from SCLC and NSCLC patients were bled into heparinized tubes and treated with Gey's solution to remove red blood cells. Patient PBMC were not processed on ficoll gradients. The resulting PBMC were used fresh.

### Flow Cytometry BiTE Binding Assays

Target cells were resuspended in HL1 medium and loaded into wells of a 96 well flat bottomed plate (2×10⁵ cells/well). Plates were centrifuged (600 g×3-5 minutes) and the supernatants removed by flicking. Twenty-five to 50 μl of medium (usually HL1) containing BiTE was added to each well and the plates incubated on ice for a minimum of 30 minutes. Cells were then washed twice by addition of 200 μl medium/well, followed by centrifugation and flicking. Thirty-five μl of anti-his antibody (ThermoScientific) was then added to each well and the plates incubated on ice for a minimum of 30 minutes. The cells were then washed twice and resuspended in 100 μl medium. If viability was tested, then 15 μl of 7AAD per 400 μl of sample was added. Labeled cells were analyzed using a Beckman Coulter Cyan ADP flow cytometer and Summit software.

### Cytotoxicity Assay

Target cells were labeled using a CellTrace Violet kit according to the manufacturer's directions (Molecular Probes). Labeled cells were adjusted to 5×10⁴ cells/ml (RPMI) and 100 μl (5,000 cells) were placed in each well of a 96-well round-bottom plate along with 100 μl of PBMC in RPMI medium at the indicated ratios. Fifty μl of HL-1 medium containing BiTE or antibodies to OKT3 or PDL1 at the desired concentration were added to each well, and the plates incubated at 30^{C}, 5% CO₂. Following a 48 hr incubation, the contents of each well were transferred to 96 well flat-bottomed plates for further analysis. If adherent target cells were used, the wells were trypsinized (50 μl trypsin-EDTA/well; trypsin neutralized with 150 μl HL-1, 5% serum), the cells washed, and added to the wells of the 96 well flat bottom plate. Resulting cells were stained with the indicated antibodies and 7AAD as previously described, and analyzed by flow cytometry on a Cyan ADP flow cytometer with Summit software. % dead tumor cells=[dead tumor cells (violet⁺7AAD⁺)/total tumor cells (violet⁺)]×100%.

### BiTE-Induced T Cell Activation and Proliferation

PBMC were resuspended to 1×10⁶ cells/ml and Violet Tracker-labeled tumor cells to 5×10⁴ cells/ml. PBMC (1×105/100±tumor cells (5000/100 μl) were incubated±BiTE or irrelevant recombinant protein (TROY-Fc) in triplicate in a total volume of 250 μl in 96-well flat bottom plates. Following a 24 or 48 hr incubation, cells and supernatants were harvested. IFNγ in supernatants was measured using an ELISA kit according to the manufacturer's directions (R&D). Cells were stained with fluorescent antibodies to CD4, CD8, CD69, and CD25. Violet-labeled tumor cells were gated out, and gated CD4⁺ and CD8⁺ T cells were assessed for expression of activation markers. Violet Tracker-labeled CD3⁺ PBMC were resuspended to 1×10⁶ cells/ml and tumor cells to 5×10⁴ cells/ml. PBMC (1×10⁵/100 μl)±tumor cells (5000/100 μl) were incubated±BiTE in a total volume of 250 μl in 96-well flat bottom plates. Following a 48 hr incubation, cells were harvested and analyzed for proliferation by flow cytometry on a Cyan ADP flow cytometer with Summit software.

### PBMC Depletions and Cell Isolations

CD4 and CD8 depletions: PBMC (107/250 μl) were incubated for 10 minutes at room temperature with 5 μg of biotinylated anti-CD4 antibody (clone RPA-T8, BioLegend), or 5 μg of biotinylated anti-CD8 antibody (clone SK3, BioLegend), washed twice with excess PBS-2% FCS, resuspended in 250 μl PBS/2% FCS and then depleted using Rapidsphere streptavidin magnetic beads (31.25 μl beads/250 μl of labeled cells) according to the manufacturer's directions (StemCell, Inc). NKT purification: For NKT experiments NKT cells were positively purified and CD3 cells were negatively purified using NKT purification kits according to the manufacturer's directions (Miltenyi Biotech). For other experiments: CD3+ PBMC were negatively purified using CD3 purification kits according to the manufacturer's directions (StemCell, Inc). Depletions and purified populations were ascertained by flow cytometry.

IFNγ induction. Cells were incubated for 48 hrs in their standard medium supplemented with 200 units/ml recombinant human IFNγ (R&D).

### In Vivo NSG Tumor Studies

NSG mice were bred and maintained in the UMBC animal facility. All animal procedures were conducted under an approved animal protocol from the UMBC Institutional Animal Care and Use Committee (IACUC). Six to eight week old NSG mice were inoculated on day 0 with 1×10⁶ C8161 melanoma cells. On day seven, when tumors were palpable, mice were adoptively transferred via the tail vein with 1×10⁷ PBMC from healthy human donors. Also on day seven, mice administered via the retro-orbital sinus 0.2 ng/mouse (8 ng/kg) CD3xPDL1 BiTE. Mice received daily BiTE treatments of 0.2 ng/mouse on days 8, 9, 10, 11, and 32. Mice were followed for survival and sacrificed when they became moribund, as required by the UMBC IACUC. Percent total CD3⁺ cells and % MDSC in the spleen=(% CD3⁺CD4⁺ plus CD3⁺CD8⁺ splenocytes as assessed by flow cytometry)×(total number of splenocytes) and %Gr1⁺CD11b⁺ splenocytes as assessed by flow cytometry)×(total number of splenocytes), respectively. Ratio of MDSC:T cells=total number of Gr1⁺CD11b⁺ splenocytes/total number of CD3⁺ splenocytes.

### Surface Plasmon Resonance (Biacore)

Biacore analysis was performed in the Biosensor Core, Dept. of Physiology, University of Maryland, Baltimore. BiTE binding to PDL1-Fc (ThermoFisher) was determined using a GE Healthcare 3000 instrument and multi-cycle kinetics. The BiTE was immobilized to a CM5 chip with anti-His antibodies and then soluble PDL1-Fc (0-5 nM) was passed over the chip. Chi square=0.264. BiTE binding to CD3 was determined using a GE Healthcare T200 instrument and single-cycle kinetics. CD3εδ heterodimer (Acro Biosystems) was immobilized to a CM5 chip using the amine coupling method in which the carboxylmethyl-dextran surface of the chip was activated with a 35 μl injection of a mixture of 0.1 M NHS and 0.1 M EDC in water. BiTE (0-10 nM) was injected without regeneration between each injection cycle. Chi square=0.421. A blank flow cell was used as a reference. The KD was calculated by using a 1:1 Langmuir kinetic model. Unless otherwise specified, all reagents were from GE Healthcare (Piscataway, N.J., USA).

### Statistical Analyses

Statistical analysis of tumor growth rates was conducted using the compare Growth Curves function of the Statmod software package (http://bioinf.wehi.edu.au/software/compareCurves/). Statistical analysis of Kaplan-Meier graphs was conducted using the Log Rank Test function of the Statmod software package (http://bioinfwehi.edu.au/software/russell/logrank/). Student's t test was utilized to determine statistical significance between two sets of data using Microsoft Excel Version 2010. p values <0.05 were considered statistically significant. One-way ANOVA with Tukey's post-hoc test was performed using GraphPad Prism version 7 for Windows, GraphPad Software, La Jolla California USA, www.graphpad.com. Error bars represent standard deviation unless noted otherwise. Asterisks in figures indicate that the experimental value is statistically significantly different from the associated controls at *=p<0.05; **=p<0.01; ***=p<0.001.

### Abbreviations

BiTE: bispecific T cell engager; CML: chronic myelogenous leukemia; i.v.: intravenous; mAb: monoclonal antibody; MDSC: myeloid-derived suppressor cells; MST: mean survival time; NSCLC: non-small cell lung cancer; PD1: programmed death 1; PDL1: programmed death ligand 1; SCLC: small cell lung cancer; s.c.: subcutaneous; sPDL1: soluble PDL1; Tregs: T regulatory cells.

Although the invention has been described with reference to the above examples, it will be understood that modifications and variations are encompassed within the spirit and scope of the invention. Accordingly, the invention is limited only by the following claims.

### REFERENCES

The contents of all references cited herein are incorporated by reference herein for all purposes.
(1) Brahmer, J R, et al., Safety and activity of anti-PDL1 antibody in patients with advanced cancer. N Engl J Med 366, 2455-2465 (2012).
(2) Topalian, S L, et al., Safety, activity, and immune correlates of anti-PD-1 antibody in cancer. N Engl J Med 366, 2443-2454 (2012).
(3) Topalian, S L, et al., Survival, durable tumor remission, and long-term safety in patients with advanced melanoma receiving nivolumab. J Clin Oncol 32, 1020-1030 (2014).
(4) Ostrand-Rosenberg, S, L A Horn & S T Haile. The Programmed Death-1 Immune-Suppressive Pathway: Barrier to Antitumor Immunity. J Immunol 193, 3835-3841 (2014).
(5) Woodle, E S, et al., Humanized OKT3 antibodies: successful transfer of immune modulating properties and idiotype expression. J Immunol 148, 2756-2763 (1992).
(6) Korman, A, et al., Human monoclonal antibodies to programmed death ligand 1 (PDL1). U.S. Pat. 20130122014 A1 (2013) (2013).
(7) Baeuerle, P A & C Reinhardt. Bispecific T-cell engaging antibodies for cancer therapy. Cancer Res 69, 4941-4944 (2009).
(8) Brahmer, J R. Immune checkpoint blockade: the hope for immunotherapy as a treatment of lung cancer? Semin Oncol 41, 126-132 (2014).
(9) Drake, C G, et al., Breathing new life into immunotherapy: review of melanoma, lung and kidney cancer. Nat Rev Clin Oncol 11, 24-37 (2014).
(10) Iclozan C, Antonia S, Chiappori A, Chen D T, Gabrilovich D. Therapeutic regulation of myeloid-derived suppressor cells and immune response to cancer vaccine in patients with extensive stage small cell lung cancer. Cancer Immunol Immunother. (2013); 62: 909-918.
(11) Srivastava M K, Bosch J J, Thompson J A, Ksander B R, Edelman M J, Ostrand-Rosenberg S. Lung cancer patients' CD4(+) T cells are activated in vitro by MHC II cell-based vaccines despite the presence of myeloid-derived suppressor cells. Cancer Immunol Immunother. (2008); 57: 1493-1504.
(12) Bronte V, Brandau S, Chen S H, Colombo M P, Frey A B, Greten T F, Mandruzzato S, Murray P J, Ochoa A, Ostrand-Rosenberg S, Rodriguez P C, Sica A, Umansky V, et al. Recommendations for myeloid-derived suppressor cell nomenclature and characterization standards. Nature Comm. (2016); 7: 12150.
(13) Jackson Laboratory. https://www.jax.org/strain/005557. 2017.
(14) Taube J M, Anders R A, Young G D, Xu H, Sharma R, McMiller T L, Chen S, Klein A P, Pardoll D M, Topalian S L, Chen L. Colocalization of inflammatory response with B7-h1 expression in human melanocytic lesions supports an adaptive resistance mechanism of immune escape. Sci Transl Med. (2012); 4: 127ra137.
(15) Hoffmann P, Hofmeister R, Brischwein K, Brandl C, Crommer S, Bargou R, Itin C, Prang N, Baeuerle P A. Serial killing of tumor cells by cytotoxic T cells redirected with a CD19-/CD3-bispecific single-chain antibody construct. Int J Cancer. (2005); 115: 98-104.
(16) Choi B D, Kuan C T, Cai M, Archer G E, Mitchell D A, Gedeon P C, Sanchez-Perez L, Pastan I, Bigner D D, Sampson J H. Systemic administration of a bispecific antibody targeting EGFRvIII successfully treats intracerebral glioma. Proc Natl Acad Sci USA. (2013); 110: 270-275.
(17) Prendergast G C, Smith C, Thomas S, Mandik-Nayak L, Laury-Kleintop L, Metz R, Muller A J. Indoleamine 2,3-dioxygenase pathways of pathogenic inflammation and immune escape in cancer. Cancer Immunol Immunother. (2014); 63: 721-735.
(18) Smith A L, Robin T P, Ford H L. Molecular pathways: targeting the TGF-beta pathway for cancer therapy. Clin Cancer Res. (2012); 18: 4514-4521.
(19) Krupka C, Kufer P, Kischel R, Zugmaier G, Lichtenegger FS, Kohnke T, Vick B, Jeremias I, Metzeler K H, Altmann T, Schneider S, Fiegl M, Spiekermann K, et al. Blockade of the PD-1/PD-L1 axis augments lysis of AML cells by the CD33/CD3 BiTE antibody construct AMG 330: reversing a T-cell-induced immune escape mechanism. Leukemia. (2016); 30: 484-491.
(20) Tanaka S, Saito Y, Kunisawa J, Kurashima Y, Wake T, Suzuki N, Shultz L D, Kiyono H, Ishikawa F. Development of mature and functional human myeloid subsets in hematopoietic stem cell-engrafted NOD/SCID/IL2rgammaKO mice. J Immunol. (2012); 188: 6145-6155.
(21) https://www.jax.org/jax-mice-and-services/find-and-order-jax-mice/nsg-portfolio/frequently-asked-nsg-questions#.
(22) Parker K H, Beury D W, Ostrand-Rosenberg S. Myeloid-Derived Suppressor Cells: Critical Cells Driving Immune Suppression in the Tumor Microenvironment. Adv Cancer Res. (2015); 128: 95-139.
(23) Ostrand-Rosenberg S, Sinha P. Myeloid-derived suppressor cells: linking inflammation and cancer. J Immunol. (2009); 182: 4499-4506.
(24) Klinger M, Benjamin J, Kischel R, Stienen S, Zugmaier G. Harnessing T cells to fight cancer with BiTE(R) antibody constructs—past developments and future directions. Immunol Rev. (2016); 270: 193-208.
(25) Lee K J, Chow V, Weissman A, Tulpule S, Aldoss I, Akhtari M. Clinical use of blinatumomab for B-cell acute lymphoblastic leukemia in adults. Ther Clin Risk Manag. (2016); 12: 1301-1310.
(26) Brandl C, Haas C, d'Argouges S, Fisch T, Kufer P, Brischwein K, Prang N, Bargou R, Suzich J, Baeuerle P A, Hofmeister R. The effect of dexamethasone on polyclonal T cell activation and redirected target cell lysis as induced by a CD19/CD3-bispecific single-chain antibody construct. Cancer Immunol Immunother. (2007); 56: 1551-1563.
(27) Dorken B, Riethmuller G, Kufer P, Lutterbuse R, Bargou R, Loffler A, inventors; Micromet Ag, assignee. CD19xCD3 specific polypeptides and uses thereof. United States patent U.S. Pat. No. 7,575,923 B2. (2009) Aug. 18.
(28) Ogura M, Morishima Y, Ohno R, Kato Y, Hirabayashi N, Nagura H, Saito H. Establishment of a novel human megakaryoblastic leukemia cell line, MEG-01, with positive Philadelphia chromosome. Blood. (1985); 66: 1384-1392.
(29) Kishi K. A new leukemia cell line with Philadelphia chromosome characterized as basophil precursors. Leuk Res. (1985); 9: 381-390.
(30) Welch D R, Bisi J E, Miller B E, Conaway D, Seftor E A, Yohem K H, Gilmore L B, Seftor R E, Nakajima M, Hendrix M J. Characterization of a highly invasive and spontaneously metastatic human malignant melanoma cell line. Int J Cancer. (1991); 47: 227-237.
(31) Haile S, Bosch J J, Agu N, Zeender A, Somasundaram P, Srivastava M K, Rodel S, Wolf J, Ksander B R, Ostrand-Rosenberg S. Tumor cell programmed death ligand-1-mediated T cell suppression is overcome by co-expression of CD80. J Immunol. (2011); 186: 6822-6829.
(32) Srivastava M K, Bosch J J, Wilson A L, Edelman M J, Ostrand-Rosenberg S. MEW II lung cancer vaccines prime and boost tumor-specific CD4+ T cells that cross-react with multiple histologic subtypes of nonsmall cell lung cancer cells. Int J Cancer. (2010); 127: 2612-2621.

That which is claimed is:

1. An anti-CD3/anti-PDL1 bispecific fusion protein comprising an anti-CD3 antibody, fragment thereof, or single chain variable fragment that binds to a CD3 receptor on a T-cell and an anti PDL1 antibody, fragment thereof, or single chain variable fragment that binds to a Programmed Death Ligand 1 (PDL1) on a tumor cell.

2. The anti-CD3/anti-PDL1 bispecific fusion protein according to claim 1, wherein the anti-CD3 antibody or fragment thereof and the anti-PDL1 antibody or fragment thereof are linked by an amino acid spacer of sufficient length of amino acid residues so that both antibody moieties can successfully bind to their individual target.

3. The anti-CD3/anti-PDL1 bispecific fusion protein according to claim 1, wherein the anti-CD3 antibody and anti-PDL1 antibody comprise both a light and heavy chain of the antibody.

4. The anti-CD3/anti-PDL1 bispecific fusion protein according to claim 3, wherein the heavy chain of the anti-PDL1 antibody is linked to the heavy chain of the anti-CD3 antibody.

5. The anti-CD3/anti-PDL1 bispecific fusion protein according to claim 3, wherein the light and heavy chains of the PDL1 antibody comprise amino acid residues SEQ ID NOs: 5 and 6 respectively and the light and heavy chains of the anti-CD3 antibody comprise amino acid residues SEQ ID NOs: 7 and 8 respectively.

6. The anti-CD3/anti-PDL1 bispecific fusion protein according to claim 2, wherein the linker comprises at least one sequence of amino acid residues selected from SEQ ID NOs. 12, 14 or 16.

7. A composition comprising the anti-CD3/anti-PDL1 bispecific fusion protein according to claim 1.

8. The composition according to claim 7, further comprising an anticancer therapy selected from the group consisting of a chemotherapeutic molecule, small molecule kinase inhibitor, hormonal agent and cytotoxic agent.

9. A method of treating cancer and/or killing cancerous tumor cells, the method comprising:
administering to a subject an anti-CD3/anti-PDL1 bispecific fusion protein comprising an anti-CD3 antibody, fragment thereof, or single chain variable fragment that binds to a CD3 receptor on a T-cell and an anti PDL1 antibody, fragment thereof, or single chain variable fragment that binds to Programmed Death Ligand 1 (PDL1) on a tumor cell, wherein the binding to the CD3 receptor on a T-cell activates CD4⁺ and CD8⁺ T-cells that are cytotoxic for a PDL1⁺ tumor and thereby inducing increased death of the PDL1⁺ tumor cell.

10. The method according to claim 9, wherein the anti-CD3 antibody or fragment thereof and the anti-PDL1 antibody or fragment thereof are linked by an amino acid spacer of sufficient length of amino acid residues so that both antibody moieties can successfully bind to their individual target.

11. The method according to claim 9, wherein the anti-CD3 antibody and anti-PDL1 antibody comprise a light and heavy chain of the antibody.

12. The method according to claim 11, wherein the heavy or light chain of the anti-PDL1 antibody is linked to the light or heavy chain of the anti-CD3 antibody.

13. The method according to claim 11, wherein the heavy and light chains of the anti-PDL1 antibody comprise amino acid residues SEQ ID NOs: 5 and 6 and the anti-CD3 antibody comprise amino acid residues SEQ ID NOs: 7 and 8.

14. The method according to claim 11, further comprising administering another anticancer therapy selected from chemotherapeutic molecule, small molecule kinase inhibitor, hormonal agent, cytotoxic agent, ionizing radiation, ultraviolet radiation, cryoablation, thermal ablation, adoptive cell therapy, hematopoietic stem cell transplant, bone marrow transplant, or radiofrequency ablation.

15. The method of claim 9, wherein the tumor cell is a PDL1⁺ human melanoma, breast cancer, AML, GBM, chronic myelogenous leukemia (CML) or non-small cell lung cancer cells.

16. A method of preparing an anti-CD3/anti-PDL1 bispecific fusion protein, the method comprising:
transfecting a host cell with polynucleotide sequences that encode the anti-CD3/anti-PDL1 bispecific fusion protein to produce a transformed host cell, wherein the polynucleotide sequences encode an anti-CD3 antibody, fragment thereof, or single chain variable fragment and an anti-PDL1 antibody, fragment thereof, or single chain variable fragment and having a nucleotide sequence of SEQ ID NO: 9; and
maintaining the transformed host cell under biological conditions sufficient for expression of the bispecific fusion protein have an amino acid sequence of SEQ ID NO: 10.

17. A method of killing cancerous tumor cells, the method comprising:
contacting a cancerous tumor cell with an anti-CD3/anti-PDL1 bispecific fusion protein comprising an anti-CD3 antibody, fragment thereof, or single chain variable fragment that binds to a CD3 receptor on a T-cell and an ant- PDL1 antibody, fragment thereof, or single chain variable fragment that binds to Programmed Death Ligand 1 (PDL1⁺) on a tumor cell, wherein the binding to the CD3 receptor on the T-cell activates CD4⁺ and CD8⁺ T-cells that are cytotoxic for a PDL1⁺ tumor cell, and thereby inducing death of the PDL1⁺ tumor cell.


SEQUENCE LISTING (16 sequences)

SEQ ID NO: 1 - DNA, 318 bp, Artificial Sequence
  Synthetic construct
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgacgttcggccaagggaccaaggtggaaatcaaa

SEQ ID NO: 2 - DNA, 369 bp, Artificial Sequence
  Synthetic construct
caggtccaactggtgcagtctggggctgaggtcaagaagcctgggtcgtcggtgaaggtctcctgcaagacttctggagacaccttcagcacctatgctatcagctgggtgcgacaggcccctggacaagggcttgagtggatgggagggatcatccctatatttggtaaagcacactacgcacagaagttccagggcagagtcacgattaccgcggacgaatccacgagcacagcctacatggagctgagcagcctgagatctgaggacacggccgtgtatttttgtgcgagaaagtttcactttgtttcggggagccccttcggtatggacgtctggggccaagggaccacggtcaccgtctcctca

SEQ ID NO: 3 - DNA, 318 bp, Artificial Sequence
  Synthetic construct
gacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaa

SEQ ID NO: 4 - DNA, 363 bp, Artificial Sequence
  Synthetic construct
gatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaa

SEQ ID NO: 5 - protein, 106 aa, Artificial Sequence
  Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 6 - protein, 123 aa, Artificial Sequence
  Synthetic construct
QVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSS

SEQ ID NO: 7 - protein, 106 aa, Artificial Sequence
  Synthetic construct
DIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 8 - protein, 121 aa, Artificial Sequence
  Synthetic construct
DIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVE

SEQ ID NO: 9 - DNA, 1533 bp, Artificial Sequence
  Synthetic construct
atgggatggagctgtatcatcctcttcttggtagcaacagctacaggtgtccactccgaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgacgttcggccaagggaccaaggtggaaatcaaaggtggaggtggatctgggggaggtggatctgggggtgggggatcgcaggtccaactggtgcagtctggggctgaggtcaagaagcctgggtcgtcggtgaaggtctcctgcaagacttctggagacaccttcagcacctatgctatcagctgggtgcgacaggcccctggacaagggcttgagtggatgggagggatcatccctatatttggtaaagcacactacgcacagaagttccagggcagagtcacgattaccgcggacgaatccacgagcacagcctacatggagctgagcagcctgagatctgaggacacggccgtgtatttttgtgcgagaaagtttcactttgtttcggggagccccttcggtatggacgtctggggccaagggaccacggtcaccgtctcctcaggaggtggtggatccgatatcaaactgcagcagtcaggggctgaactggcaagacctggggcctcagtgaagatgtcctgcaagacttctggctacacctttactaggtacacgatgcactgggtaaaacagaggcctggacagggtctggaatggattggatacattaatcctagccgtggttatactaattacaatcagaagttcaaggacaaggccacattgactacagacaaatcctccagcacagcctacatgcaactgagcagcctgacatctgaggactctgcagtctattactgtgcaagatattatgatgatcattactgccttgactactggggccaaggcaccactctcacagtctcctcagtcgaaggtggaagtggaggttctggtggaagtggaggttcaggtggagtcgacgacattcagctgacccagtctccagcaatcatgtctgcatctccaggggagaaggtcaccatgacctgcagagccagttcaagtgtaagttacatgaactggtaccagcagaagtcaggcacctcccccaaaagatggatttatgacacatccaaagtggcttctggagtcccttatcgcttcagtggcagtgggtctgggacctcatactctctcacaatcagcagcatggaggctgaagatgctgccacttattactgccaacagtggagtagtaacccgctcacgttcggtgctgggaccaagctggagctgaaa

SEQ ID NO: 10 - protein, 492 aa, Artificial Sequence
  Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKGGGGSGGGGSGGGGSQVQLVQSGAEVKKPGSSVKVSCKTSGDTFSTYAISWVRQAPGQGLEWMGGIIPIFGKAHYAQKFQGRVTITADESTSTAYMELSSLRSEDTAVYFCARKFHFVSGSPFGMDVWGQGTTVTVSSGGGGSDIKLQQSGAELARPGASVKMSCKTSGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSVEGGSGGSGGSGGSGGVDDIQLTQSPAIMSASPGEKVTMTCRASSSVSYMNWYQQKSGTSPKRWIYDTSKVASGVPYRFSGSGSGTSYSLTISSMEAEDAATYYCQQWSSNPLTFGAGTKLELK

SEQ ID NO: 11 - DNA, 45 bp, Artificial Sequence
  Synthetic construct
ggtggaggtggatctgggggaggtggatctgggggtgggggatcg

SEQ ID NO: 12 - protein, 15 aa, Artificial Sequence
  Synthetic Construct
GGGGSGGGGSGGGGS

SEQ ID NO: 13 - DNA, 15 bp, Artificial Sequence
  Synthetic construct
ggaggtggtggatcc

SEQ ID NO: 14 - protein, 5 aa, Artificial Sequence
  Synthetic construct
GGGGS

SEQ ID NO: 15 - DNA, 48 bp, Artificial Sequence
  Synthetic construct
ggtggaagtggaggttctggtggaagtggaggttcaggtggagtcgac

SEQ ID NO: 16 - protein, 16 aa, Artificial Sequence
  Synthetic construct
GGSGGSGGSGGSGGVD